SEQUENCE LISTING (14 sequences)

SEQ ID NO: 1 - RNA, 7 nt, Artificial Sequence
  Synthetic Oligonucleotide
nuununu

SEQ ID NO: 2 - RNA, 7 nt, Artificial Sequence
  Synthetic Oligonucleotide
guunugu

SEQ ID NO: 3 - RNA, 7 nt, Artificial Sequence
  Synthetic Oligonucleotide
nuugugu

SEQ ID NO: 4 - RNA, 7 nt, Artificial Sequence
  Synthetic Oligonucleotide
guugunu

SEQ ID NO: 5 - RNA, 7 nt, Artificial Sequence
  Synthetic Oligonucleotide
nuugugu

SEQ ID NO: 6 - RNA, 7 nt, Artificial Sequence
  Synthetic Oligonucleotide
nuugugu

SEQ ID NO: 7 - RNA, 8 nt, Artificial Sequence
  Synthetic Oligonucleotide
nguugugu

SEQ ID NO: 8 - RNA, 8 nt, Artificial Sequence
  Synthetic Oligonucleotide
gauugugu

SEQ ID NO: 9 - RNA, 20 nt, Artificial Sequence
  Synthetic Oligonucleotide
gccaccgagccgaaggcacc

SEQ ID NO: 10 - RNA, 7 nt, Artificial Sequence
  Synthetic Oligonucleotide
nuugugu

SEQ ID NO: 11 - RNA, 18 nt, Artificial Sequence
  Synthetic Oligonucleotide
ccgucuguugugugacuc

SEQ ID NO: 12 - RNA, 20 nt, Artificial Sequence
  Synthetic Oligonucleotide
uuguuguuguuguuguuguu

SEQ ID NO: 13 - DNA, 22 bp, Artificial Sequence
  Synthetic Oligonucleotide
tcgtcgttttcggcgcgcgccg

SEQ ID NO: 14 - DNA, 19 bp, Artificial Sequence
  Synthetic Oligonucleotide
tccatgacgttcctgatgc

(19) United States
(12) Patent Application Publication
(10) Pub. No.: US 2010/0189772 A1
(43) Pub. Date: Jul. 29, 2010

(54) Compositions of TLR ligands and antivirals

(75) Inventors: Jorg Vollmer, Dusseldorf (DE); Marion Jurk, Dormagen (DE); Eugen Uhlmann, Glashuetten (DE); Harald Debelak, Hilden (DE); Robert L. Bratzler, Concord, MA (US); Alain Vicari, Neydens (FR)

Correspondence Address: PHARMACIA & UPJOHN, 7000 Portage Road, KZO-300-104, KALAMAZOO, MI, 49001, US

(73) Assignees: COLEY PHARMACEUTICAL GROUP, INC, New York, NY (US); COLEY PHARMACEUTICAL GMBH, Duesseldorf (DE); COLEY PHARMACEUTICAL GROUP, LTD, Ottawa, ON (CA)

(21) Appl. No.: 12/443,224

(22) PCT Filed: Sep. 27, 2007
(86) PCT No.: PCT/US07/21030

§ 371 Date: Jan. 25, 2010

Related U.S. Application Data
(60) Provisional application No. 60/847,408, filed on Sep. 27, 2006.

(51) Int. Cl. A61K 9/127; C07H 21/02; C07H 21/04; A61K 39/12; A61P 31/04; A61P 31/12

(52) U.S. Cl. 424/450; 536/23.1; 424/204.1

(57) ABSTRACT

The invention relates to methods and products for the treatment of viral infection using a combination of anti-viral agents and TLR ligands. The invention also relates to screening assays, associated products, kits, and in vitro methods.

### FIELD OF THE INVENTION

The present invention relates generally to compositions composed of TLR ligands and antivirals and their use in methods such as the treatment of viral infections and screening assays.

### BACKGROUND OF THE INVENTION

Toll-like receptors (TLRs) are a family of highly conserved pattern recognition receptor (PRR) polypeptides that recognize pathogen-associated molecular patterns (PAMPs) and play a critical role in innate immunity in mammals. Currently at least ten family members, designated TLR1-TLR10, have been identified. The cytoplasmic domains of the various TLRs are characterized by a Toll-interleukin 1 receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adapter protein MyD88 has been reported to associate with TLRs and to recruit interleukin 1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For reviews, see Aderem A et al. (2000) Nature 406:782-87, and Akira S et al. (2004) Nat Rev Immunol 4:499-511.

Recently certain low molecular weight synthetic compounds, the imidazoquinolines imiquimod (R-837) and resiquimod (R-848), were reported to be ligands of TLR7 and TLR8. Hemmi H et al. (2002) Nat Immunol 3:196-200; Jurk M et al. (2002) Nat Immunol 3:499.

Beginning with the recent discovery that unmethylated bacterial DNA and synthetic analogs thereof (CpG DNA) are ligands for TLR9 (Hemmi H et al. (2000) Nature 408:740-5; Bauer S et al. (2001) Proc Natl Acad Sci USA 98, 9237-42), it has been reported that ligands for certain TLRs include certain nucleic acid molecules. Recently it has been reported that certain types of RNA are immunostimulatory in a sequence-independent or sequence-dependent manner. Further, it has been reported that these various immunostimulatory RNAs stimulate TLR3, TLR7, or TLR8. In addition, certain low molecular weight synthetic compounds, the imidazoquinolines imiquimod (R-837) and resiquimod (R-848), were reported to be ligands of TLR7 and TLR8. Hemmi H et al. (2002) Nat Immunol 3:196-200; Jurk M et al. (2002) Nat Immunol 3:499. Viral-derived double-stranded RNA (dsRNA) and poly I:C, a synthetic analog of dsRNA, were recently reported to be ligands of TLR3. Alexopoulou L et al. (2001) Nature 413:732-8. Even more recently, Lipford and coworkers disclosed that certain G,U-containing RNA sequences are immunostimulatory, acting through stimulation of both TLR7 and TLR8. Heil F et al. (2004) Science 303:1526-9, and U.S. Pat. Appl. 2003/0232074 A1.

Heil et al. reported that guanosine- and uridine-rich phosphorothioate ssRNA oligonucleotides, derived from HIV-1 and complexed with the cationic lipid DOTAP, stimulate dendritic cells (DC) and macrophages to secrete interferon alpha (IFN-α), tumor necrosis factor (TNF), interleukin 12 (IL-12), and interleukin 6 (IL-6). Heil F et al. (2004) Science 303:1526-9. Murine TLR7 was reported to confer responsiveness to GU-rich ssRNA, and human TLR8 was reported to confer responsiveness to GU-rich and U-rich ssRNA. Although specific sequences were tested, no motif was identified. Ibid.

Diebold et al. recently reported that single-stranded RNA (ssRNA) of viral or synthetic origin activates TLR7. Diebold S S et al. (2004) Science 303:1529-31. They reported that viral genomic ssRNA from influenza virus, as well as polyU, triggers IFN-α production by plasmacytoid dendritic cells (pDC). No sequence-specific motif was identified beyond polyU. Mouse spleen and some short ssRNA oligos (of the type used to make short interfering dsRNA) also induced IFN-α. Ibid.

### SUMMARY OF THE INVENTION

Methods and products for the prevention and/or treatment of viral infections are provided according to the invention. In one aspect the invention is a composition of an immunostimulatory oligonucleotide and an anti-viral agent, wherein the anti-viral agent is not a C-8 substituted guanosine and wherein the anti-viral agent is linked to the immunostimulatory oligonucleotide.

The immunostimulatory oligonucleotide may be an RNA oligonucleotide (ORN) or a DNA oligonucleotide (ODN). The DNA oligonucleotide, in some embodiments is an A-class, B-class, C-Class, P-class, T-class, or E class oligonucleotide and optionally may include at least one unmethylated CpG dinucleotide. In other embodiment the DNA oligonucleotide includes at least three unmethylated CpG dinucleotides. The at least one, two or three unmethylated CpG dinucleotides may includes a phosphodiester or phosphodiester-like internucleotide linkage, and wherein the oligonucleotide includes at least one stabilized internucleotide linkage. In other embodiments the immunostimulatory oligonucleotide comprises a chimeric backbone.

A composition of an immunostimulatory RNA oligonucleotide and an anti-viral agent wherein the anti-viral agent is associated with the immunostimulatory RNA oligonucleotide is provided according to other aspects of the invention.

The immunostimulatory oligonucleotide may be linked to the anti-viral agent indirectly or directly. In one embodiment the immunostimulatory oligonucleotide and the anti-viral agent are part of the same molecule. The antiviral agent may be linked to an internal nucleotide or a terminal nucleotide, optionally a 3′ terminal nucleotide or a 5′ terminal nucleotide.

The composition may include a nuclease susceptible site between immunostimulatory oligonucleotide and the anti-viral agent.

In some embodiments the immunostimulatory oligonucleotide contains at least one 3′-3′ linkage and/or 5′-5′ linkage.

The composition may include a pharmaceutically acceptable carrier. In some embodiments the composition is sterile.

The anti-viral agent may be, for instance, one or more nucleotide analogues, loxoribine, isatoribine, ribavirin, valopicitabine, BILN 2061, VX-950.

In some embodiments the composition includes a second anti-viral agent formulated with the immunostimulatory oligonucleotide. The second anti-viral agent may be linked to the immunostimulatory oligonucleotide. In other embodiments the composition includes a microparticle or liposome housing the immunostimulatory oligonucleotide and the anti-viral agents.

In some embodiments the anti-viral agent is a C-8 substituted guanosine. The C-8 substituted guanosine may be incorporated in the RNA oligonucleotide or it may be linked to the RNA. In some embodiments the C-8 substituted guanosine is positioned at the 5′ end of the RNA oligonucleotide. In other embodiments the C-8 substituted guanosine is positioned one, two or three nucleotides 3′ of the 5′ end of the RNA oligonucleotide.

In some embodiments the DNA oligonucleotide is not an abasic containing oligonucleotide or an adapter oligonucleotide.

In other aspects the invention is a composition of a TLR7/8/9 ligand linked to an anti-viral agent. In some embodiments the TLR7/8/9 ligand is an immunostimulatory oligonucleotide. The TLR7/8/9 ligand is linked to the anti-viral agent directly or indirectly. In some embodiments the composition includes a nuclease susceptible site between the TLR7/8/9 ligand and the anti-viral agent.

A method for treating viral disease is provided according to other aspects of the invention. The method involves administering to a subject in need of such treatment a composition of the invention described herein in an amount effective to treat the viral disease. In some embodiments the viral disease is human immunodeficiency virus (HIV), hepatitis C virus (HCV), or hepatitis B virus (HBV). The carrier may be a buffer.

In some embodiments the subject is non-responsive to a non-CpG therapy. In other embodiments the subject is non-responsive to therapy with the anti-viral agent.

A composition of a cell capable of expressing an inhibitory viral protein and a TLR and a carrier is provided according to other aspects of the invention.

In one embodiment the cell is transfected with a TLR reporter construct. The TLR may be TLR 7, TLR 8, or TLR9.

In another embodiment the cell is transfected with an inhibitory viral protein expression construct. The inhibitory viral protein may be for instance NS3/4 protease.

In some embodiments the cell is an immune cell from a virally infected patient.

In other embodiments the inhibitory viral protein is endogenously expressed by the cell.

A method for identifying an immune-stimulating anti-viral composition, is provided according to other aspects of the invention. The method involves contacting a cell described herein with a test compound and measuring cytokine production and anti-viral reporter readout, wherein an increase in cytokine production and an increase in anti-viral reporter readout indicates that the test compound is an immune-stimulating anti-viral composition.

In yet other aspects the invention is a method for identifying an immune-stimulating anti-viral composition, comprising contacting a cell described herein with a test compound and measuring a Th1 response, a Th-1-like response, or pro-inflammatory cytokine production, wherein an increase in a Th1 response, a Th-1-like response, or pro-inflammatory cytokine production indicates that the test compound is an immune-stimulating anti-viral composition.

In yet another aspect the invention is a method for identifying an immune-stimulating anti-viral composition, by isolating immune cells from a virus-infected patient, contacting the cells with a test compound and measuring cytokine production and viral titer, wherein an increase in Th1 cytokine production and a decrease in viral titer indicates that the test compound is an immune-stimulating anti-viral composition.

In other aspects the invention is a method for screening for molecules containing an anti-viral agent and an immunostimulatory oligonucleotide that have anti-viral activity, by isolating immune cells from a virus-infected patient, contacting the cells with the molecule and measuring viral titer, wherein a reduction in viral titer indicates that the molecule has anti-viral activity.

In some embodiments the peripheral blood mononuclear cells comprise dendritic cells. The dendritic cells may be plasmacytoid dendritic cells.

The step of contacting may occur in vitro and the peripheral blood mononuclear cells may be cultured.

Use of a composition of the invention for stimulating an immune response is also provided as an aspect of the invention.

A method for manufacturing a medicament of a composition of the invention for stimulating an immune response is also provided.

A method for treating cancer is also provided according to aspects of the invention. The method involves administering to a subject having cancer a composition of an immunostimulatory oligonucleotide and an anti-viral agent in an amount effective to treat the cancer.

In another aspects the invention includes a method for treating bacterial infection by administering to a subject having a bacterial infection a composition of an immunostimulatory oligonucleotide and an anti-viral agent in an amount effective to treat the bacterial infection.

In some embodiments the anti-viral agent is linked to the immunostimulatory oligonucleotide. The anti-viral agent may be ribavirin. The composition, may also include a C-8 substituted guanosine.

In some embodiments the immunostimulatory oligonucleotide is an RNA oligonucleotide. In other embodiments the immunostimulatory oligonucleotide is a DNA oligonucleotide such as an A-class, B-class, C-Class, P-class, T-class, or E-class oligonucleotide. The DNA oligonucleotide includes at least one unmethylated CpG dinucleotide.

A composition as described herein for treating a cancer, or a viral or bacterial infection is also provided.

Use of a composition as provided herein in combination with an antigen, for the manufacture of a medicament for vaccinating a subject is also provided.

The invention also includes use of a composition as provided herein for the manufacture of a medicament for treating cancer, viral infection or a bacterial infection in a subject.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS

FIG. 1 is three graphs demonstrating the positive impact of 8-Oxo-rG on ORN-mediated immune stimulation. Cytokine stimulation by 8-Oxo-rG modified ORN (SEQ ID NO:1 and SEQ ID NO:8) was compared to that of the control ORN (SEQ ID NO:11). Cytokines IFN-alpha (FIG. 1a), IL-12p40 (FIG. 1b) and TNF-alpha (FIG. 1c) were measured. The x-axes are ORN concentration in μM and the y-axes are cytokine concentration in pg/ml.

FIG. 2 is a graph demonstrating that the positive impact of 8-modified G depends on position in the RNA sequence. IFN-alpha stimulation by URN with a single 8-Oxo-rG at different positions of the ORN (SEQ ID NO:1-4) and an unmodified ORN (SEQ ID NO:8). The x-axis is ORN concentration in μM and the y-axis is IFN-alpha concentration in pg/ml.

FIG. 3 is a graph demonstrating that Different 8-modified deoxy- and ribonucleotides at the ORN 5′ end increase the immune stimulatory activity. IFN-alpha stimulation by ORN with a single 8-Oxo-rG/Dg (SEQ ID NO:1, 5), 8-Bromo-dG (SEQ ID NO:7) or Immunosine (Isatoribine) (SEQ ID NO:6) (with a 5′-5′ linkage) at the 5′ end of the ORN was compared to the 8-Bromo-dA modified ORN (SEQ ID NO:10), the control ORN SEQ ID NO:11, and an unmodified ORN (SEQ ID NO:8) (FIG. 3). The x-axis is ORN concentration in μM and the y-axis is IFN-alpha concentration in pg/ml.

FIG. 4 is a set of graphs depicting the effects of combination of RBV and CpG ODN (SEQ ID No. 14) T cell IFN-γ production (FIG. 4B) and RBV on IFN-γ production in the absence of CpG ODN (FIG. 4A).

FIG. 5 is a set of graphs depicting the ex vivo effect of RBV on CD3-mediated IFN-γ production independently of prior ODN/RBV treatment. Low concentrations of RBV in vitro increased IFN-γ levels independently of previous in vivo treatments (FIG. 5A). The effect of a combination with CpG ODN (SEQ ID No. 14) is shown in FIG. 5B.

FIG. 6 is a graph demonstrating that RBV decreased SEQ ID NO. 14-induced IL-10.

FIG. 7 is a set of graphs depicting an experiment performed using bone marrow (BM) derived dendritic cells (DCs). BM-derived DC maintained in GM-CSF were treated with SEQ ID NO. 14, RBV (1 μM, 5 μM, 10 μM, 100 μM, or 120 μM) or with SEQ ID NO. 14 and RBV and tested for IL-12p40 (FIG. 7A), IL-12p70 (FIG. 7B).

FIG. 8 is a graph depicting the results of an in vivo study on the effects of a combination of CpG ODN (SEQ ID NO. 14) and RBV in a mouse cancer model.

### DETAILED DESCRIPTION

The invention relates to methods and products for the treatment of viral infection, bacterial infection or cancer using a combination of anti-viral agents and TLR ligands such as immunostimulatory oligonucleotides. The invention also includes in vitro assays using the combination of agents.

Coadministration of the composition can be accomplished either by combining the components into one molecule or in a delivery vehicle that will deliver them simultaneously to the target cell. The combined TLR ligands and anti-viral agents of the invention are useful for the treatment of viral disorders, such as acute viral infections or chronic viral infections. Acute viral infection refers to a short course of infection, generally less than 6 months that may self-resolve. A chronic infection is one that recurs or lasts longer than 6 months in duration and requires intervention for resolution.

Viruses are small infectious agents which generally contain a nucleic acid core and a protein coat, but are not independently living organisms. Viruses can also take the form of infectious nucleic acids lacking a protein. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and others are RNA-containing viruses. DNA viruses include Pox, Herpes, Adeno, Papova, Parvo, and Hepadna. RNA viruses include Picorna, Calici, Astro, Toga, Flavi, Corona, Paramyxo, Orthomyxo, Bunya, Arena, Rhabdo, Filo, Borna, Reo, and Retro. In some aspects, the invention also intends to treat diseases in which prions are implicated in disease progression such as for example bovine spongiform encephalopathy (i.e., mad cow disease, BSE) or scrapie infection in animals, or Creutzfeldt-Jakob disease in humans.

Viruses include, but are not limited to, enteroviruses (including, but not limited to, viruses that the family picornaviridae, such as polio virus, Coxsackie virus, echo virus), rotaviruses, adenovirus, and hepatitis virus, such as hepatitis A, B, C D and E. Specific examples of viruses that have been found in humans include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papillomaviruses, polyoma viruses); Adenoviridae (most adenoviruses); Herpesviridae (herpes simplex virus (HSV) 1 and 2, varicella zoster virus, cytomegalovirus (CMV)); Poxyiridae (variola viruses, vaccinia viruses, pox viruses); Iridoviridae (e.g., African swine fever virus); and other viruses acute laryngotracheobronchitis virus, Alphavirus, Kaposi's sarcoma-associated herpesvirus, Newcastle disease virus, Nipah virus, Norwalk virus, Papillomavirus, parainfluenza virus, avian influenza, SARs virus, West Nile virus.

The methods of the invention are particularly useful, in some embodiments, for the treatment of Human immunodeficiency virus (HIV) and hepatitis virus. HIV, a species of retrovirus also known as human T-cell lymphotropic virus III (HTLV III), is responsible for causing the deterioration resulting in the disorder known as AIDS. HIV infects and destroys T-cells, upsetting the overall balance of the immune system, resulting in a loss in the patients ability to combat other infections and predisposing the patient to opportunistic infections which frequently prove fatal.

Viral hepatitis is an inflammation of the liver which may produce swelling, tenderness, and sometimes permanent damage to the liver. If the inflammation of the liver continues at least six months or longer, it is referred to as chronic hepatitis. There are at least five different viruses known to cause viral hepatitis, include hepatitis A, B, C D and E. Hepatitis A is generally communicated through food or drinking water contaminated with human feces. Hepatitis B generally is spread through bodily fluids such as blood. For instance, it may be spread from mother to child at birth, through sexual contact, contaminated blood transfusions and needles. Hepatitis C is quite common and like Hepatitis B is often spread through blood transfusions and contaminated needles. Hepatitis D is found most often in IV drug users who are carriers of the hepatitis B virus with which it co-associates. Hepatitis E is similar to viral hepatitis A and is generally associated with poor sanitation.

As used herein, the term “subject” refers to a human or non-human vertebrate. Non-human vertebrates include livestock animals, companion animals, and laboratory animals. Non-human subjects also specifically include non-human primates as well as rodents. Non-human subjects also specifically include, without limitation, chickens, horses, cows, pigs, goats, dogs, cats, guinea pigs, hamsters, mink, and rabbits. In some embodiments the subject is a patient. As used herein, a “patient” refers to a subject who is under the care of a physician other health care worker, including someone who has consulted with, received advice from or received a prescription or other recommendation from a physician or other health care worker. A patient is typically a subject having or at risk of having a viral infection.

A “subject having a viral infection” is a subject that has or is at risk of having a disorder arising from the invasion of the subject, superficially, locally, or systemically, by an infectious virus. A subject at risk of having a viral infection may be someone known to be exposed to a particular virus, such as those traveling to places where the virus is known to be found, those living in places where the virus is known to be found, and those in close proximity to someone known to be infected with a virus. The method for treating a viral infection in a subject having or at risk of developing a viral infection according to the invention involves administering to a subject in need, of such treatment a composition of the invention in an effective amount for treating the viral infection.

The TLR ligand-antiviral agent compositions function in some aspects by simultaneously inducing innate and antigen specific immune responses leading to a multifaceted attack by the immune system on the virus. The anti-viral agents specifically attack the virus, while the immunostimulatory oligonucleotides provide long-lasting effects. The combination is designed to reduce dosing regimes, improve compliance and maintenance therapy, reduce emergency situations; and improve quality of life.

TLR ligands stimulate the immune system to treat viral infection. The strong yet balanced, cellular and humoral immune responses that result from the immune stimulatory capacity of the oligonucleotide reflect the natural defense system of the subject against invading viruses. As used herein, the term “treat” as used in reference to a disease or condition shall mean to intervene in such disease or condition so as to prevent or slow the development of, prevent, inhibit, or slow the progression of, halt the progression of, or eliminate the disease or condition. As used herein, the term “inhibit” shall mean reduce an outcome or effect compared to normal.

The compositions of the invention include TLR ligands linked to one or more antiviral agents. Toll-like receptors (TLRs) are a family of highly conserved polypeptides that play a critical role in innate immunity in mammals. Currently ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adaptor protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For a review, see Aderem A et al. (2000) Nature 406:782-87.

TLRs are believed to be differentially expressed in various tissues and on various types of immune cells. For example, human TLR7 has been reported to be expressed in placenta, lung, spleen, lymph nodes, tonsil and on plasmacytoid precursor dendritic cells (pDCs). Chuang T-H et al. (2000) Eur Cytokine Netw 11:372-8); Kadowaki N et al. (2001) J Exp Med 194:863-9. Human TLR8 has been reported to be expressed in lung, peripheral blood leukocytes (PBL), placenta, spleen, lymph nodes, and on monocytes. Kadowaki N et al. (2001) J Exp Med 194:863-9; Chuang T-H et al. (2000) Eur Cytokine Netw 11:372-8. Human TLR9 is reportedly expressed in spleen, lymph nodes, bone marrow, PBL, and on pDCs, and B cells. Kadowaki N et al. (2001) J Exp Med 194:863-9; Bauer S et al. (2001) Proc Natl Acad Sci USA 98:9237-42; Chuang T-H et al. (2000) Eur Cytokine Netw 11:372-8.

Nucleotide and amino acid sequences of human and murine TLR7 are known. See, for example, GenBank Accession Nos. AF240467, AF245702, NM_{—}016562, AF334942, NM_{—}133211; and AAF60188, AAF78035, NP_{—}057646, AAL73191, and AAL73192, the contents of all of which are incorporated herein by reference. Human TLR7 is reported to be 1049 amino acids long. Murine TLR7 is reported to be 1050 amino acids long. TLR7 polypeptides include an extracellular domain having a leucine-rich repeat region, a transmembrane domain, and an intracellular domain that includes a TIR domain.

Nucleotide and amino acid sequences of human and murine TLR8 are known. See, for example, GenBank Accession Nos. AF246971, AF245703, NM_{—}016610, XM_{—}045706, AY035890, NM_{—}133212; and AAF64061, AAF78036, NP_{—}057694, XP_{—}045706, AAK62677, and NP_{—}573475, the contents of all of which is incorporated herein by reference. Human TLR8 is reported to exist in at least two isoforms, one 1041 amino acids long and the other 1059 amino acids long. Murine TLR8 is 1032 amino acids long. TLR8 polypeptides include an extracellular domain having a leucine-rich repeat region, a transmembrane domain, and an intracellular domain that includes a TIR domain.

Nucleotide and amino acid sequences of human and murine TLR9 are known. See, for example, GenBank Accession Nos. NM_{—}017442, AF259262, AB045180, AF245704, AB045181, AF348140, AF314224, NM_{—}031178; and NP_{—}059138, AAF72189, BAB19259, AAF78037, BAB19260, AAK29625, AAK28488, and NP_{—}112455, the contents of all of which are incorporated herein by reference. Human TLR9 is reported to exist in at least two isoforms, one 1032 amino acids long and the other 1055 amino acids. Murine TLR9 is 1032 amino acids long. TLR9 polypeptides include an extracellular domain having a leucine-rich repeat region, a transmembrane domain, and an intracellular domain that includes a TIR domain.

As used herein, the term “TLR signaling” refers to any aspect of intracellular signaling associated with signaling through a TLR. As used herein, the term “TLR-mediated immune response” refers to the immune response that is associated with TLR signaling.

A TLR7-mediated immune response is a response associated with TLR7 signaling. TLR7-mediated immune response is generally characterized by the induction of IFN-α and IFN-inducible cytokines such as IP-10 and I-TAC. The levels of cytokines IL-1 α/β, IL-6, IL-8, MIP-1α/β and MIP-3α/β induced in a TLR7-mediated immune response are less than those induced in a TLR8-mediated immune response.

A TLR8-mediated immune response is a response associated with TLR8 signaling. This response is further characterized by the induction of pro-inflammatory cytokines such as IFN-γ, IL-12p40/70, TNF-α, IL-1α/β, IL-6, IL-8, MIP-1α/β and MIP-3 α/β.

A TLR9-mediated immune response is a response associated with TLR9 signaling. This response is further characterized at least by the production/secretion of IFN-γ and IL-12, albeit at levels lower than are achieved via a TLR8-mediated immune response.

As used herein, a “TLR7/8 ligand” or “TLR7/8 agonist” collectively refers to any agent that is capable of increasing TLR7 and/or TLR8 signaling (i.e., an agonist of TLR7 and/or TLR8). Some TLR7/8 ligands induce TLR7 signaling alone (e.g., TLR7 specific ligands), some induce TLR8 signaling alone (e.g., TLR8 specific ligands), and others induce-both TLR7 and TLR8 signaling.

As used herein, the term “TLR7 ligand” or “TLR7 agonist” refers to any agent that is capable of increasing TLR7 signaling (i.e., an agonist of TLR7). In this respect, the level of TLR7 signaling may be enhanced over a pre-existing level of signaling or it may be induced over a background level of signaling. TLR7 ligands include, without limitation, guanosine analogues such as C8-substituted guanosines, mixtures of ribonucleosides consisting essentially of G and U, guanosine ribonucleotides and RNA or RNA-like molecules (PCT/US03/10406), and adenosine-based compounds (e.g., 6-amino-9-benzyl-2-(3-hydroxy-propoxy)-9H-purin-8-ol, and similar compounds made by Sumitomo (e.g., CL-029)). TLR7 ligands are also disclosed in Gorden et al. J. Immunol. 2005, 174:1259-1268 (e.g., 3M-001, N-[4-(4-amino-2-ethyl-1H-imidazo[4,5-c]quinolin-1-yl)butyl-]methanesulfonamide; C17H23N5O2S; mw 361).

As used herein, the term “guanosine analogues” refers to a guanosine-like nucleotide (excluding guanosine) having a chemical modification involving the guanine base, guanosine nucleoside sugar, or both the guanine base and the guanosine nucleoside sugar. Guanosine analogues specifically include, without limitation, 7-deaza-guanosine.

Guanosine analogues further include C8-substituted guanosines such as 7-thia-8-oxoguanosine (immunosine), 8-mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7-substituted guanine ribonucleosides such as 7-allyl-8-oxoguanosine (loxoribine) and 7-methyl-8-oxoguanosine, 8-aminoguanosine, 8-hydroxy-2′-deoxyguanosine, -8-hydroxyguanosine, and 7-deaza 8-substituted guanosine.

As used herein, the term “TLR8 ligand” or “TLR8 agonist” refers to any agent that is capable of increasing TLR8 signaling (i.e., an agonist of TLR8). In this respect, the level of TLR8 signaling may be enhanced over a pre-existing level of signaling or it may be induced over a background level of signaling. TLR8 ligands include mixtures of ribonucleosides consisting essentially of G and U, guanosine ribonucleotides and RNA or RNA-like molecules (PCT/US03/10406). Additional TLR8 ligands are also disclosed in Gorden et al. J. Immunol. 2005, 174:1259-1268).

Some TLR7/8 ligands are ligands of both TLR7 and TLR8. These include imidazoquinolines, mixtures of ribonucleosides consisting essentially of G and U, guanosine ribonucleotides and RNA or RNA-like molecules (PCT/US03/10406). Additional TLR7/8 ligands are also disclosed in Gorden et al. J. Immunol. 2005, 174:1259-1268 (e.g., 3M 003, 4-amino-2-(ethoxymethyl)-α,α-dimethyl-6,7,8,9-tetrahydro-1H-imidazo[4,5-c]quinoline-1-ethanol hydrate, C17H26N4O2; mw 318).

Imidazoquinolines are immune response modifiers thought to induce expression of several cytokines including interferons (e.g., IFN-α), TNF-α and some interleukins (e.g., IL-1, IL-6 and IL-12). Imidazoquinolines are capable of stimulating a Th1 immune response, as evidenced in part by their ability to induce increases in IgG2a levels. Imidazoquinoline agents reportedly are also capable of inhibiting production of Th2 cytokines such as IL-4, IL-5, and IL-13. Some of the cytokines induced by imidazoquinolines are produced by macrophages and dendritic cells. Some species of imidazoquinolines have been reported to increase NK cell lytic activity and to stimulate B cells proliferation and differentiation, thereby inducing antibody production and secretion.

As used herein, an imidazoquinoline agent includes imidazoquinoline amines, imidazopyridine amines, 6,7-fused cycloalkylimidazopyridine amines, and 1,2 bridged imidazoquinoline amines. These compounds have been described in U.S. Pat. Nos. 4,689,338, 4,929,624, 5,238,944, 5,266,575, 5,268,376, 5,346,905, 5,352,784, 5,389,640, 5,395,937, 5,494,916, 5,482,936, 5,525,612, 6,039,969 and 6,110,929. Particular species of imidazoquinoline agents include R-848 (S-28463); 4-amino-2ethoxymethyl-α,α-dimethyl-1H-imidazo[4,5-c]quinolines-1-ethanol; 1-(2-methylpropyl)-1H-imidazo[4,5-c]quinolin-4-amine (R-837 or Imiquimod), and S-27609. Imiquimod is currently used in the topical treatment of warts such as genital and anal warts and has also been tested in the topical treatment of basal cell carcinoma.

As used herein, the term “TLR9 ligand” or “TLR9 agonist” refers to any agent that is capable of increasing TLR9 signaling (i.e., an agonist of TLR9). TLR9 ligands specifically include, without limitation, immunostimulatory nucleic acids, and in particular CpG immunostimulatory nucleic acids.

As used herein, the term “immunostimulatory CpG nucleic acids” or “immunostimulatory CpG oligonucleotides” refers to any CpG-containing nucleic acid that is capable of activating an immune cell. At least the C of the CpG dinucleotide is typically, but not necessarily, unmethylated. Immunostimulatory CpG nucleic acids are described in a number of issued patents and published patent applications, including U.S. Pat. Nos. 6,194,388; 6,207,646; 6,218,371; 6,239,116; 6,339,068; 6,406,705; and 6,429,199.

In some embodiments the TLR ligand is an immunostimulatory oligonucleotide. An “immunostimulatory oligonucleotide” as used herein is any nucleic acid (DNA or RNA) containing an immunostimulatory motif or backbone that is capable of inducing an immune response. An induction of an immune response refers to any increase in number or activity of an immune cell, or an increase in expression or absolute levels of an immune factor, such as a cytokine. Immune cells include, but are not limited to, NK cells, CD4+ T lymphocytes, CD8+ T lymphocytes, B cells, dendritic cells, macrophage and other antigen-presenting cells. Cytokines include, but are not limited to, interleukins, TNF-α, IFN-α,β and γ, Flt-ligand, and co-stimulatory molecules. Immunostimulatory motifs include, but are not limited to CpG motifs and T-rich motifs. Immunostimulatory backbones include, but are not limited to, phosphate modified backbones, such as phosphorothioate backbones. Immunostimulatory oligonucleotides have been described extensively in the prior art and a brief summary of these nucleic acids is presented below.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis).

The oligonucleotides can be double-stranded or single-stranded. In certain embodiments, while the oligonucleotide is single stranded, it is capable of forming secondary and tertiary structures (e.g., by folding back on itself, or by hybridizing with itself either throughout its entirety or at select segments along its length). Accordingly, while the primary structure of such an oligonucleotide may be single stranded, its higher order structures may be double or triple stranded.

Immunostimulatory oligonucleotides may possess immunostimulatory motifs such as unmethylated CpG motifs and non-CpG motifs such as T-rich motifs. Depending upon the embodiment of the invention, some immunostimulatory motifs are preferred over others. In some embodiments of the instant invention, the immunostimulatory oligonucleotides do not contain poly-G motifs. In some embodiments, any nucleic acid, regardless of whether it possesses an identifiable motif, can be combined with the anti-viral agent. Immunostimulatory oligonucleotides also include nucleic acids having a modified backbone, such as a phosphorothioate modified backbone. In particular embodiments, the immunostimulatory oligonucleotides having a phosphorothioate modified backbone does not also have an identifiable motif, yet it is still immunostimulatory.

CpG sequences, while relatively rare in human DNA, are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG containing nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570), PCT/US97/19791, PCT/US98/03678; PCT/US98/10408; PCT/US98/04703; PCT/US99/07335; and PCT/US99/09863.

A “CpG oligonucleotide” is a nucleic acid which includes at least one unmethylated CpG dinucleotide. In some embodiments, the nucleic acid includes three or more unmethylated CpG dinucleotides. A nucleic acid containing at least one “unmethylated CpG dinucleotide” is a nucleic acid molecule which contains an unmethylated cytosine in a cytosine-guanine dinucleotide sequence (i.e. “CpG DNA” or DNA containing a 5′ cytosine followed by 3′ guanosine and linked by a phosphate bond) and activates the immune system.

For facilitating uptake into cells, the immunostimulatory oligonucleotides are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

The immunostimulatory oligonucleotides may contain a palindrome or inverted repeat (i.e. a sequence such as ABCDEE′D′C′B′A′ in which A and A′ are bases capable of forming the usual Watson-Crick base pairs). In vivo, such sequences may form double-stranded structures. In one embodiment the CpG nucleic acid contains a palindromic sequence. A palindromic sequence used in this context refers to a palindrome in which the CpG is part of the palindrome, and preferably is the center of the palindrome. In another embodiment the CpG nucleic acid is free of a hexameric palindrome. An immunostimulatory nucleic acid that is free of a hexameric palindrome is one in which the CpG dinucleotide is not part of a palindrome that is at least 6 nucleotides in length. Such an oligonucleotide may include a palindrome in which the CpG is not within the palindrome.

In some embodiments of the invention, a non-CpG immunostimulatory nucleic acid is used. A non-CpG immunostimulatory nucleic acid is a nucleic acid that does not have a CpG motif in its sequence, regardless of whether the C residue of the dinucleotide is methylated or unmethylated. Non-CpG immunostimulatory oligonucleotides may induce Th1 or Th2 immune responses, depending upon their sequence, their mode of delivery, and the dose at which they are administered.

In select aspects of the invention, the non-CpG immunostimulatory oligonucleotides may be T-rich nucleic acids. T-rich nucleic acids are nucleic acids having T-rich motifs. T rich motifs and nucleic acids possessing such motifs are described in U.S. patent application Ser. No. 09/669,187, filed Sep. 25, 2000, by Krieg et al., the entire contents of which are incorporated herein by reference. Other non-CpG nucleic acids useful in the present invention are described in U.S. patent application Ser. No. 09/768,012, filed Jan. 22, 2001, the entire contents of which are incorporated herein in their entirety by reference

In some embodiments the immunostimulatory oligonucleotides have a modified backbone such as a phosphorothioate backbone. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner. Thus, some embodiments of the invention rely on the use of phosphorothioate backbone oligonucleotides that lack methylated and unmethylated CpG and T-rich motifs.

The methods of the invention may embrace the use of previously described classes of immunostimulatory oligonucleotides including ODN classes such as A class, B class, C class, E class, T class and P class. In some embodiments of the invention the immunomodulatory oligonucleotides include immunostimulatory motifs which are “CpG dinucleotides”. A CpG dinucleotide can be methylated or unmethylated. An immunostimulatory nucleic acid containing at least one unmethylated CpG dinucleotide is a nucleic acid molecule which contains an unmethylated cytosine-guanine dinucleotide sequence (i.e., an unmethylated 5′ cytidine followed by 3′ guanosine and linked by a phosphate bond) and which activates the immune system; such an immunostimulatory nucleic acid is a CpG nucleic acid. CpG nucleic acids have been described in a number of issued patents, published patent applications, and other publications, including U.S. Pat. Nos. 6,194,388; 6,207,646; 6,214,806; 6,218,371; 6,239,116; and 6,339,068. An immunostimulatory nucleic acid containing at least one methylated CpG dinucleotide is a nucleic acid which contains a methylated cytosine-guanine dinucleotide sequence (i.e., a methylated 5′ cytidine followed by a 3′ guanosine and linked by a phosphate bond) and which activates the immune system. In other embodiments the immunostimulatory oligonucleotides are free of CpG dinucleotides. These oligonucleotides which are free of CpG dinucleotides are referred to as non-CpG oligonucleotides, and they have non-CpG immunostimulatory motifs. Preferably these are T-rich ODN, such as ODN having at least 80% T.

“B class” ODN are potent at activating B cells but are relatively weak in inducing IFN-α and NK cell activation. The B class CpG nucleic acids typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class is potent for inducing IFN-α and NK cell activation but is relatively weak at stimulating B cells; this class has been termed the “A class”. The A class CpG nucleic acids typically have stabilized poly-G sequences at 5′ and 3′ ends and a palindromic phosphodiester CpG dinucleotide-containing sequence of at least 6 nucleotides. See, for example, published patent application PCT/US00/26527 (WO 01/22990). Yet another class of CpG nucleic acids activates B cells and NK cells and induces IFN-α; this class has been termed the C-class.

The “C class” immunostimulatory nucleic acids contain at least two distinct motifs have unique and desirable stimulatory effects on cells of the immune system. Some of these ODN have both a traditional “stimulatory” CpG sequence and a “GC-rich” or “B-cell neutralizing” motif. These combination motif nucleic acids have immune stimulating effects that fall somewhere between those effects associated with traditional “class B” CpG ODN, which are strong inducers of B cell activation and dendritic cell (DC) activation, and those effects associated with a more recently described class of immune stimulatory nucleic acids (“class A” CpG ODN) which are strong inducers of IFN-α and natural killer (NK) cell activation but relatively poor inducers of B-cell and DC activation. Krieg A M et al. (1995) Nature 374:546-9; Ballas Z K et al. (1996) J Immunol 157:1840-5; Yamamoto S et al. (1992) J Immunol 148:4072-6. While preferred class B CpG ODN often have phosphorothioate backbones and preferred class A CpG ODN have mixed or chimeric backbones, the C class of combination motif immune stimulatory nucleic acids may have either stabilized, e.g., phosphorothioate, chimeric, or phosphodiester backbones, and in some preferred embodiments, they have semi-soft backbones. This class has been described in U.S. patent application U.S. Ser. No. 10/224,523 filed on Aug. 19, 2002, the entire contents of which is incorporated herein by reference.

The “P class” immunostimulatory oligonucleotides have several domains, including a 5′TLR activation domain, 2 duplex forming regions and an optional spacer and 3′ tail. This class of oligonucleotides has the ability in some instances to induce much higher levels of IFN-α secretion than the C-Class. The P-Class oligonucleotides have the ability to spontaneously self-assemble into concatamers either in vitro and/or in vivo. Without being bound by any particular theory for the method of action of these molecules, one potential hypothesis is that this property endows the P-Class oligonucleotides with the ability to more highly crosslink TLR9 inside certain immune cells, inducing a distinct pattern of immune activation compared to the previously described classes of CpG oligonucleotides. Cross-linking of TLR9 receptors may induce activation of stronger IFN-α secretion through the type I IFNR feedback loop in plasmacytoid dendritic cells. P class oligonucleotides are described at least in U.S. application Ser. No. 11/706,561.

The “T class” oligonucleotides induce secretion of lower levels of IFN-alpha when not modified as in the ODNs of the invention and IFN-related cytokines and chemokines than B class or C class oligonucleotides, while retaining the ability to induce levels of IL-10 similar to B class oligonucleotides. T class oligonucleotides are described at least in U.S. Published patent application Ser. No. 11/099,683, the entire contents of which are hereby incorporated by reference.

The “E class” oligonucleotides have an enhanced ability to induce secretion of IFN-alpha. These ODN have a lipophilic substituted nucleotide analog 5′ and/or 3′ of a YGZ motif. The compound of the E class formula may be, for example, any of the following lipophilic substituted nucleotide analogs: a substituted pyrimidine, a substituted uracil, a hydrophobic T analog, a substituted toluene, a substituted imidazole or pyrazole, a substituted triazole, 5-chloro-uracil, 5-bromo-uracil, 5-iodo-uracil, 5-ethyl-uracil, 5-propyl-uracil, 5-propinyl-uracil, (E)-5-(2-bromovinyl)-uracil, or 2,4-difluoro-toluene. E class oligonucleotides are described at least in provisional patent application U.S. 60/847,811.

In some embodiments of the invention the immunostimulatory oligonucleotide is an oligoribonucleotide (ORN). Immunostimulatory ORNs include for instance, those that stimulate TLR7/8 motifs. A TLR7/8 stimulating ORN may include for example a ribonucleotide sequence such as 5′-C/U-U-G/U-U-3′,5′-R-U-R-G-Y-3′, 5′-G-U-U-G-B-3′,5′-G-U-G-U-G/U-3′, or 5′-G/C-U-A/C-G-G-C-A-C-3′. C/U is cytosine (C) or uracil (U), G/U is guanine (G) or U, R is purine, Y is pyrimidine, B is U, G, or C, G/C is G or C, and A/C is adenine (A) or C. The 5′-C/U-U-G/U-U-3′ may be CUGU, CUUU, UUGU, or UUUU. In various embodiments 5′-R-U-R-G-Y-3′ is GUAGU, GUAGC, GUGGU, GUGGC, AUAGU, AUAGC, AUGGU, or AUGGC. In one embodiment the base sequence is GUAGUGU. In various embodiments 5′-G-U-U-G-B-3′ is GUUGU, GUUGG, or GUUGC. In various embodiments 5′-G-U-G-U-G/U-3′ is GUGUG or GUGUU. In one embodiment the base sequence is GUGUUUAC. In various other embodiments 5′-G/C-U-A/C-G-G-C-A-C-3′ is GUAGGCAC, GUCGGCAC, CUAGGCAC, or CUCGGCAC.

In some embodiments the oligonucleotides are not adapter oligonucleotides or abasic oligonucleotides.

Adaptor oligonucleotides comprise the formula 5′Xₐ-TTTTT-X_{b} 3′, wherein Xₐ and X_{b} can independently be any nucleotide and may be present or absent. Xₐ and X_{b} represent one or more nucleotides (e.g., 1-100 nucleotides). The oligonucleotide may be 7, 8, 9, 10, 11, 12, 13, 14, 15, 16, 17, 18, 19, 20, 21, 22, 23 or more nucleotides in length. The oligonucleotide may comprise 6, 7 or more contiguous T. Preferably, the adaptor oligonucleotide is a dT homopolymer (i.e., oligo dT of a length recited herein). Even more preferably, the adaptor oligonucleotide is a thymidine (dT) homopolymer 17 nucleotides in length. Most preferably, it comprises at least one phosphorothioated internucleotide linkage (up to and including a completely phosphorothioated backbone).

The adaptor oligonucleotide may be comprised of 100% T, 99% T, 98% T, 97% T, 96% T, 95% T, 94% T, 93% T, 92% T, 91% T, 90% T, 85% T, 80% T, 75% T, 70% T, 65% T, 60% T, 55% T, 50% T, 45% T or less, depending on the embodiment.

Another class of adaptor oligonucleotides comprises the formula 5′ Xₐ-UUUUU-X_{b} 3′ wherein Xₐ and X_{b} can independently be any nucleotide and may be present or absent. Xₐ and X_{b} represent one or more nucleotides (e.g., 1-100 nucleotides). The oligonucleotide may be 7, 8, 9, 10, 11, 12, 13, 14, 15, 16, 17, 18, 19, 20, 21, 22, 23 or more nucleotides in length. The oligonucleotide may comprise 6, 7 or more contiguous U. In an important embodiment, the oligonucleotide is a dU homopolymer that is preferably 17 nucleotides in length and having at least one phosphorothioated internucleotide linkage (up to an including a completely phosphorothioated backbone).

The adaptor oligonucleotide may be comprised of 100% U, 99% U, 98% U, 97% U, 96% U, 95% U, 94% U, 93% U, 92% U, 91% U, 90% U, 85% U, 80% U, 75% U, 70% U, 65% U, 60% U, 55% U, 50% U, 45% U or less, depending on the embodiment.

Yet, another class of adaptor oligonucleotides comprises the formula 5′ Xₐ-AAAAA-X_{b} 3′ wherein Xₐ and X_{b} can independently be any nucleotide and may be present or absent. Xₐ and X_{b} represent one or more nucleotides (e.g., 1-100 nucleotides). The oligonucleotide may be 7, 8, 9, 10, 11, 12, 13, 14, 15, 16, 17, 18, 19, 20, 21, 22, 23 or more nucleotides in length. The oligonucleotide may comprise 6, 7 or more contiguous A. In an important embodiment, the oligonucleotide is a dA homopolymer that is preferably 17 nucleotides in length and having at least one phosphorothioated internucleotide linkage (up to an including a completely phosphorothioated backbone).

The adaptor oligonucleotide may be comprised of 100% A, 99% A, 98% A, 97% A, 96% A, 95% A, 94% A, 93% A, 92% A, 91% A, 90% A, 85% A, 80% A, 75% A, 70% A, 65% A, 60% A, 55% A, 50% A, 45% A or less, depending on the embodiment.

Another class of adaptor oligonucleotides comprises the formula 5′ Cₙ-Tₘ-Cₚ 3′, wherein n is an integer ranging from 0-100 (e.g., 3-7), p is an integer ranging from 0-100 (e.g., 4-8), and m is an integer ranging from 0-100 (e.g., 2-10). Preferably, the sum of n and p is equal to or less than the value of m such that C content is less than 60%, less than 55%, less than 50%, less than 45%, less than 40%, less than 35%, less than 30%, less than 25%, less than 20%, less than 15%, less than 10%, less than 5%, or less. In some embodiments, n ranges from 3-7, m ranges from 2-10 and p ranges from 4-8, provided the percentages cited above are satisfied.

An abasic oligonucleotide resembles a backbone of a DNA or an RNA molecule, wherein the nucleobases (e.g., adenine, cytosine, thymine, uracil, and guanine) and optionally the sugar residues are absent. The abasic oligonucleotide is thus a polymer of units connected by phosphate-containing linkages. Each unit of the polymeric abasic oligonucleotide includes a phosphate group, or a thioated derivative thereof, covalently linked to an organic residue which contains at least three carbon atoms. The organic residue comprises an alkyl group, either linear or cyclic, being saturated or unsaturated, which can contain O, N and S heteroatoms, and in addition can include substituents containing C, H, N, O, S, halogen atoms, and any combination thereof.

The organic residue is preferably derived from propane-1,3-diol or sugar residues, such as β-D-deoxyribofuranose or β-D-ribofuranose. Other residues include butane-1,4-diol, triethylene glycol units, or hexaethylene glycol units ((OCH₂CH₂)ₚO, where p is 3 or 6), hydroxyl-alkyl-amino linkers, such as C3, C6, C12 aminolinkers, and also alkylthiol linkers, such as C3 or C6 thiol linkers. The sugar derivatives can also contain ring expansions, such as pyranose.

The abasic oligonucleotide can also contain a Doubler or Trebler unit (Glen Research, Sterling, Va.), in particular comprising a 3′3′-linkage. Branching of the oligonucleotides by multiple doubler, trebler, or other multiplier units leads to dendrimers which are a further embodiment of this invention.

A unit can be an abasic deoxyribonucleotide represented as

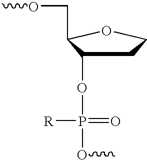

wherein R represents oxygen, sulfur, methyl, or O-alkyl.

A unit can be an abasic ribonucleotide represented as

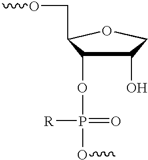

wherein R represents oxygen, sulfur, methyl, or O-alkyl.

A unit can be a C3 spacer/phosphate represented as

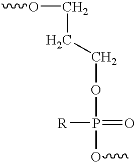

wherein R represents oxygen, sulfur, methyl, or O-alkyl.

The abasic oligonucleotide may be a homopolymer of abasic deoxyribonucleotides (poly-D). Each unit in this embodiment includes an abasic 2′-deoxyribose sugar residue and a 5′ phosphate group. In another embodiment the abasic oligonucleotide is a homopolymer of abasic ribonucleotides. Each unit in this embodiment includes an abasic 2′-hydroxyribose sugar residue and a 5′ phosphate group. In another embodiment the abasic oligonucleotide is a heteropolymer of abasic ribonucleotides and abasic deoxyribonucleotides.

The immunostimulatory oligonucleotides useful according to the invention may have modified backbones. For example, they may comprise at least one internucleotide linkage which is not a phosphodiester linkage. Such a linkage may be a phosphorothioate linkage. In some embodiments, the oligonucleotides may have chimeric backbones (i.e., backbones comprised of at least two different types of internucleotide linkages).

As used herein, the term “phosphorothioate backbone” refers to a stabilized sugar phosphate backbone of an oligonucleotide in which a non-bridging phosphate oxygen is replaced by sulfur at least one internucleotide linkage. In one embodiment a non-bridging phosphate oxygen is replaced by sulfur at each and every internucleotide linkage.

The oligonucleotides of the instant invention can encompass various chemical modifications and substitutions, in comparison to natural RNA and DNA, involving a phosphodiester internucleoside bridge, a β-D-ribose unit and/or a natural nucleoside base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-29; and Hunziker J et al. (1995) Mod Synth Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

For example, the oligonucleotides may include one or more modifications and wherein each modification is independently selected from:
a) the replacement of a phosphodiester internucleoside bridge located at the 3′ and/or the 5′ end of a nucleoside by a modified internucleoside bridge,
b) the replacement of phosphodiester bridge located at the 3′ and/or the 5′ end of a nucleoside by a dephospho bridge,
c) the replacement of a sugar phosphate unit from the sugar phosphate backbone by another unit,
d) the replacement of a β-D-ribose unit by a modified sugar unit, and
e) the replacement of a natural nucleoside base by a modified nucleoside base.

More detailed examples for the chemical modification of an oligonucleotide follow.

The oligonucleotides may include modified internucleotide linkages, such as those described in a or b above. These modified linkages may be partially resistant to degradation (e.g., are stabilized). A “stabilized oligonucleotide molecule” shall mean an oligonucleotide that is relatively resistant to in vivo degradation (e.g., via an exo- or endo-nuclease) resulting from such modifications. Oligonucleotides having phosphorothioate linkages, in some embodiments, may provide maximal activity and protect the oligonucleotide from degradation by intracellular exo- and endo-nucleases.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)—O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)-aryl, and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleoside by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine. The oligonucleotide may have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker may be branched or unbranched, substituted or unsubstituted, and chirally pure or a racemic mixture.

The β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, 2′-O-methylribose, 2′-O—(C₂-C₆)alkenyl-ribose, 2′-[O-(C₁-C₆)alkyl-O-(C₁-C₆)alkyl]-ribose, 2′—NH₂-2′-deoxyribose, β-xylo-furanose, α-arabinofuranose, 2,4-dideoxy-β-D-erythro-hexo-pyranose, and carbocyclic (described, for example, in Froehler (1992) J Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481). In some embodiments, the modified sugar is a 2′ modified ribose.

In some embodiments the sugar is 2′-O-methylribose, particularly for one or both nucleotides linked by a phosphodiester or phosphodiester-like internucleoside linkage.

Nucleic acids also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleoside base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleosides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleoside bases. This list is meant to be exemplary and is not to be interpreted to be limiting.

In particular formulas described herein modified bases may be incorporated. For instance a cytosine may be replaced with a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g., 3-nitropyrrole, P-base), an aromatic ring system (e.g., fluorobenzene or difluorobenzene) or a hydrogen atom (dSpacer).

A guanine may be replaced with a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g., N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g., N6-methyl-adenine, 8-oxo-adenine), 8-substituted guanine (e.g., 8-hydroxyguanine and 8-bromoguanine), and 6-thioguanine. In another embodiment of the invention, the guanine base is substituted by a universal base (e.g., 4-methyl-indole, 5-nitro-indole, and K-base), an aromatic ring system (e.g., benzimidazole or dichloro-benzimidazole, 1-methyl-1H-[1,2,4]triazole-3-carboxylic acid amide) or a hydrogen atom (dSpacer).

For use in the instant invention, the oligonucleotides of the invention can be synthesized de novo using any of a number of procedures well known in the art, for example, the β-cyanoethyl phosphoramidite method (Beaucage S L et al. (1981) Tetrahedron Lett 22:1859); or the nucleoside H-phosphonate method (Garegg et al. (1986) Tetrahedron Lett 27:4051-4; Froehler B C et al. (1986) Nucleic Acids Res 14:5399-407; Garegg et al. (1986) Tetrahedron Lett 27:4055-8; Gaffney et al. (1988) Tetrahedron Lett 29:2619-22). These chemistries can be performed by a variety of automated nucleic acid synthesizers available in the market. These oligonucleotides are referred to as synthetic oligonucleotides. An isolated oligonucleotide generally refers to an oligonucleotide which is separated from components which it is normally associated with in nature. As an example, an isolated oligonucleotide may be one which is separated from a cell, from a nucleus, from mitochondria or from chromatin.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann E et al. (1990) Chem Rev 90:544; Goodchild J (1990) Bioconjugate Chem 1:165).

In some embodiments the oligonucleotides may be soft or semi-soft oligonucleotides. A soft oligonucleotide is an immunostimulatory oligonucleotide having a partially stabilized backbone, in which phosphodiester or phosphodiester-like internucleotide linkages occur only within and immediately adjacent to at least one internal pyrimidine-purine dinucleotide (YZ). Preferably YZ is YG, a pyrimidine-guanosine (YG) dinucleotide. The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n−1 dinucleotides and only n−3 internal dinucleotides. Each internucleotide linkage in an internal dinucleotide is an internal internucleotide linkage. Thus a linear oligonucleotide that is n nucleotides long has a total of n−1 internucleotide linkages and only n−3 internal internucleotide linkages. The strategically placed phosphodiester or phosphodiester-like internucleotide linkages, therefore, refer to phosphodiester or phosphodiester-like internucleotide linkages positioned between any pair of nucleotides in the nucleic acid sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal YZ dinucleotide is itself an internal internucleotide linkage. Thus for a sequence N₁ YZ N₂, wherein N₁ and N₂ are each, independent of the other, any single nucleotide, the YZ dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides according to the instant invention are believed to be relatively susceptible to nuclease cleavage compared to completely stabilized oligonucleotides. Without meaning to be bound to a particular theory or mechanism, it is believed that soft oligonucleotides of the invention are cleavable to fragments with reduced or no immunostimulatory activity relative to full-length soft oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

A semi-soft oligonucleotide is an immunostimulatory oligonucleotide having a partially stabilized backbone, in which phosphodiester or phosphodiester-like internucleotide linkages occur only within at least one internal pyrimidine-purine (YZ) dinucleotide. Semi-soft oligonucleotides generally possess increased immunostimulatory potency relative to corresponding fully stabilized immunostimulatory oligonucleotides. Due to the greater potency of semi-soft oligonucleotides, semi-soft oligonucleotides may be used, in some instances, at lower effective concentrations and have lower effective doses than conventional fully stabilized immunostimulatory oligonucleotides in order to achieve a desired biological effect.

It is believed that the foregoing properties of semi-soft oligonucleotides generally increase with increasing “dose” of phosphodiester or phosphodiester-like internucleotide linkages involving internal YZ dinucleotides. Thus it is believed, for example, that generally for a given oligonucleotide sequence with five internal YZ dinucleotides, an oligonucleotide with five internal phosphodiester or phosphodiester-like YZ internucleotide linkages is more immunostimulatory than an oligonucleotide with four internal phosphodiester or phosphodiester-like YG internucleotide linkages, which in turn is more immunostimulatory than an oligonucleotide with three internal phosphodiester or phosphodiester-like YZ internucleotide linkages, which in turn is more immunostimulatory than an oligonucleotide with two internal phosphodiester or phosphodiester-like YZ internucleotide linkages, which in turn is more immunostimulatory than an oligonucleotide with one internal phosphodiester or phosphodiester-like YZ internucleotide linkage. Importantly, inclusion of even one internal phosphodiester or phosphodiester-like YZ internucleotide linkage is believed to be advantageous over no internal phosphodiester or phosphodiester-like YZ internucleotide linkage. In addition to the number of phosphodiester or phosphodiester-like internucleotide linkages, the position along the length of the nucleic acid can also affect potency.

The soft and semi-soft oligonucleotides will generally include, in addition to the phosphodiester or phosphodiester-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate, or methylphosphorothioate internucleotide linkage. In another embodiment, the degradation-resistant end includes one or more nucleotide units connected by peptide or amide linkages at the 3′ end.

A phosphodiester internucleotide linkage is the type of linkage characteristic of nucleic acids found in nature. As shown in FIG. 20, the phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNAse H. Thus for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diasteromerically pure Rp phosphorothioate. It is believed that diasteromerically pure Rp phosphorothioate is more susceptible to nuclease digestion and is better at activating RNAse H than mixed or diastereomerically pure Sp phosphorothioate. Stereoisomers of CpG oligonucleotides are the subject of co-pending U.S. patent application Ser. No. 09/361,575 filed Jul. 27, 1999, and published PCT application PCT/US99/17100 (WO 00/06588). It is to be noted that for purposes of the instant invention, the term “phosphodiester-like internucleotide linkage” specifically excludes phosphorodithioate and methylphosphonate internucleotide linkages.

As described above the soft and semi-soft oligonucleotides of the invention may have phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and S_{P} stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rp may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

A surprisingly strong effect is achieved by the p-chirality at the CpG dinucleotide itself. In comparison to a stereo-random CpG oligonucleotide the congener in which the single CpG dinucleotide was linked in Rp was slightly more active, while the congener containing an Sp linkage was nearly inactive for inducing spleen cell proliferation.

In each of the foregoing aspects of the invention, the composition can also further include a pharmaceutically acceptable carrier, such that the invention also provides pharmaceutical compositions containing the TLR ligands and antiviral agent of the invention.

The compositions of the invention can also be used for the preparation of a medicament for use in treatment of a viral condition in a subject. The use according to this aspect of the invention involves the step of placing an effective amount of a composition of the invention in a pharmaceutically acceptable carrier.

In certain embodiments the TLR ligands and antiviral agent are isolated. An isolated molecule is a molecule that is substantially pure and is free of other substances with which it is ordinarily found in nature or in in vivo systems to an extent practical and appropriate for its intended use. In particular, the agents are sufficiently pure and are sufficiently free from other biological constituents of cells so as to be useful in, for example, producing pharmaceutical preparations. Because an isolated agent of the invention may be admixed with a pharmaceutically acceptable carrier in a pharmaceutical preparation, the agent(s) may comprise only a small percentage by weight of the preparation. The agent is nonetheless isolated in that it has been substantially separated from the substances with which it may be associated in living systems.

As used herein, an “anti-viral agent” is a compound which prevents infection of cells by viruses or replication of the virus within the cell. There are many fewer anti-viral drugs than antibacterial drugs because the process of viral replication is so closely related to DNA replication within the host cell, that non-specific anti-viral agents would often be toxic to the host. There are several stages within the process of viral infection which can be blocked or inhibited by anti-viral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Nucleoside analogues are synthetic compounds which are similar to nucleotides, but which have an incomplete or abnormal deoxyribose or ribose group. Once the nucleotide analogues are in the cell they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Several US patents describe anti-viral compounds. For instance, U.S. Pat. No. 7,094,768 describes -hydroxyamino- or a 6-alkoxyamino-7-deazapurine-ribofuranose derivatives for treating HCV; U.S. Pat. No. 7,041,698 describes tripeptide compounds, compositions and methods for the treatment of HCV; U.S. Pat. No. 6,995,174 describes HCV inhibitors; U.S. Pat. No. 7,022,736 describes Diketoacids as viral inhibitors; U.S. Pat. No. 6,909,000 describes bridged bicyclic HCV NS3-NS4A serine protease inhibitors; U.S. Pat. No. 6,867,185 describes macrocyclic inhibitors of HCV; U.S. Pat. No. 6,869,964 describes Heterocyclicsulfonamide HCV inhibitors; U.S. Pat. No. 6,846,810 describes Antiviral nucleoside derivatives; and Published PCT No.: WO 0248157 describes imidazolidinones and their related derivatives as HCV NS3 Protease Inhibitors

Several drugs have been or are being developed to block entry of a virus into a host cell. These include amantadine and rimantadine, which are used against influenza; pleconaril for treatment of rhinoviruses, enteroviruses, meningitis, conjunctivitis, and encephalitis.

As mentioned above, nucleotide or nucleoside analogues are a class of drugs that target the processes that synthesize virus components after a virus invades a cell. Aciclovir, is a nucleoside analogue that is effective against herpesvirus infections. Zidovudine (AZT), for treating HIV, is also a nucleoside analogue. Lamivudine is used to treat hepatitis B, which uses reverse transcriptase as part of its replication process.

Other anti-virals being developed include targets of Rnase H and integrase, compounds based on ribozymes, protease inhibitors and drugs that interfere with the release of viruses from the host cell such as zanamivir and oseltamivir for the treatment of influenza.

Examples of anti-virals currently being used include:

Lamivudine (2′,3′-dideoxy-3′-thiacytidine, 3TC) used for treatment of HIV and chronic hepatitis B is a reverse transcriptase inhibitor marketed by GlaxoSmithKline under the brand names Epivir® and Epivir-HBV®. It is also called 3TC. It is an analogue of cytidine.

Abacavir (ABC) is a nucleoside analog reverse transcriptase inhibitor (NARTI) used to treat HIV and AIDS. It is available under the trade name Ziagen™ (GlaxoSmithKline} and the combination drugs Trizivir™ (abacavir, zidovudine and lamivudine) and Kivexa®/Epzicom™ (abacavir and lamivudine). ABC is an analog of guanosine (a purine). Its target is the viral reverse transcriptase enzyme.

Aciclovir (INN) or acyclovir (USAN), chemical name acycloguanosine, is a guanine analogue antiviral drug used for the treatment of, for example, Herpes simplex virus type I (HSV-1), Herpes simplex virus type II (HSV-2), Varicella zoster virus (VZV), Epstein-Barr virus (EBV), and Cytomegalovirus (CMV). It is one of the most commonly-used antiviral drugs, and is most commonly marketed under the trade name Zovirax (GSK). Aciclovir differs from previous nucleoside analogues in that it contains only a partial nucleoside structure—the sugar ring is replaced by an open-chain structure. Aciclo-GTP is a very potent inhibitor of viral DNA polymerase.

Amantadine (1-aminoadamantane, sold as Symmetrel®) is an antiviral drug for the treatment of Influenzavirus A.

Didanosine (2′-3′-dideoxyinosine, ddI) is sold under the trade names Videx® and Videx EC®. It is a reverse transcriptase inhibitor, effective against HIV and used in combination with other antiretroviral drug therapy as part of highly active antiretroviral therapy (HAART). Didanosine (ddI) is a nucleoside analogue of adenosine having hypoxanthine attached to the sugar ring.

Emtricitabine (FTC), with trade name Emtriva® (formerly Coviracil), is a nucleoside reverse transcriptase inhibitor (NRTI) for the treatment of HIV infection in adults. Emtricitabine is an analogue of cytidine.

Enfuvirtide (INN) is an HIV fusion inhibitor, marketed under the trade name Fuzeon (Roche).

Entecavir is an oral antiviral drug used in the treatment of hepatitis B infection, marketed under the trade name Baraclude (BMS). Entecavir is a guanine analogue that inhibits all three steps in the viral replication process

Ganciclovir is an antiviral medication used to treat or prevent cytomegalovirus (CMV) infections. Ganciclovir is a synthetic analogue of 2′-deoxy-guanosine.

Nevirapine, also marketed under the trade name Viramune® (Boehringer Ingelheim), is a non-nucleoside reverse transcriptase inhibitor (NNRTI) used to treat HIV-1 infection and AIDS but is a protease inhibitor.

Oseltamivir is an antiviral drug that is used in the treatment and prophylaxis of both Influenzavirus A and Influenzavirus B. It is a neuraminidase inhibitor acting as a transition-state analogue inhibitor of influenza neuraminidase, preventing new viruses from emerging from infected cells. Oseltamivir is indicated for the treatment of infections due to influenza A and B virus as well as against canine parvovirus, feline panleukopenia, the canine respiratory complex known as “kennel cough,” and the emerging disease dubbed “canine flu”.

Ribavirin (Copegus®; Rebetol®; Ribasphere®; Vilona®, Virazole®, also generics from Sandoz, Teva, Warrick) is an anti-viral drug which is active against a number of DNA and RNA viruses. It is a member of the nucleoside antimetabolite drugs that interfere with duplication of viral genetic material. Ribavirin has a wide range of activity, including important activities against influenzas, flaviviruses and agents of many viral hemorrhagic fevers hepatitis C, respiratory syncytial virus-related diseases and influenza. In one embodiment, administration of ribavirin with TLR7,8,9 ligands such as CpG ODNs or ORNs lowers the amount of IL-10 relative to IFN-alpha produced as a result of the TLR ligand.

AICA-Riboside is an anti-viral drug similar to Ribavirin. In one embodiment, administration of AICA-Riboside with TLR7,8,9 ligands such as CpG ODNs or ORNs lowers the amount of IL-10 relative to IFN-alpha produced as a result of the TLR ligand.

Rimantadine trade name Flumadine® is an orally administered medicine used to treat, and in rare cases prevent, Influenzavirus A infection.

Stavudine (2′-3′-didehydro-2′-3′-dideoxythymidine, d4T, brand name Zerit®) is a nucleoside analog reverse transcriptase inhibitor (NARTI) active against HIV. Stavudine is an analog of thymidine.

Valaciclovir (INN) or valacyclovir (USAN) is an antiviral drug used in the management of herpes simplex and herpes zoster (shingles).

Vidarabine is an anti-viral drug which is active against herpes simplex and varicella zoster viruses. Vidarabine (9-β-D-ribofuranosyladenine) is an analog of adenosine with the D-ribose sugar, replaced with D-arabinose.

Zalcitabine (2′-3′-dideoxycytidine, ddC), also called dideoxycytidine, is a nucleoside analog reverse transcriptase inhibitor (NARTI) sold under the trade name Hivid®. Zalcitabine is an analog of pyrimidine.

In some aspects of the invention an anti-viral agent such as a nucleoside analogue may be incorporated into the immunostimulatory oligonucleotide during synthesis of the oligonucleotide at one or various positions on the molecule, such as the 3′ or 5′ termini. This may also include incorporation of nuclease susceptible sites at the side of the nucleoside analogue(s) to allow for cleavage of the anti-viral compound after administration to allow for its anti-viral activity independent of the immunostimulatory oligonucleotide. The anti-viral agent can also be linked by other linkages (e.g., 3′-3′) or linkers (e.g., non-nucleotide linkers) to the immune stimulatory ON.

In addition conjugation of ligands for different TLRs into one molecule may lead to multimerisation of receptors which results in enhanced immune stimulation or a different immunostimulatory profile from that resulting from any single such ligand.

The invention provides a composition including a TLR ligand linked to an anti-viral agent. As used herein, the term “linked” refers to any combination of two or more component parts that are linked together, directly or indirectly, via any physicochemical interaction. In one embodiment the linkage is a combination of two or more component parts that are linked together, directly or indirectly, via covalent bonding. Thus, in some embodiments, the TLR ligands of the invention can be administered together with, but physically separate from, the anti-viral agents. However in other embodiments, ligand-anti-viral agent conjugates are contemplated.

The linkers may be attached to any reactive moiety on the oligonucleotide including but not limited to a backbone phosphate group or a sugar hydroxyl group. For example, they may be incorporated via phosphodiester, phosphorothioate, methylphosphonate and/or amide linkages. The different molecules are synthesized by established methods and can be linked together on-line during solid-phase synthesis. Alternatively, they may be linked together following synthesis of the individual partial sequences.

The linkers may be non-nucleotide in nature. Non-nucleotidic linkers are e.g. abasic residues (dSpacer), oligoethyleneglycol, such as triethyleneglycol (spacer 9) or hexaethylenegylcol (spacer 18), or alkane-diol, such as butanediol. The spacer units are preferably linked by phosphodiester or phosphorothioate bonds. The linker units may appear just once in the molecule or may be incorporated several times, e.g. via phosphodiester, phosphorothioate, methylphosphonate, or amide linkages. Further preferred linkers are alkylamino linkers, such as C3, C6, C12 aminolinkers, and also alkylthiol linkers, such as C3 or C6 thiol linkers. The oligonucleotides can also be linked by aromatic residues which may be further substituted by alkyl or substituted alkyl groups. The oligonucleotides may also contain a Doubler or Trebler unit, which allow conjugation of multiple ligands of one or different types to the oligonucleotide. The oligonucleotides may also contain linker units resulting from peptide modifying reagents or oligonucleotide modifying reagents (www.glenres.com). Furthermore, it may contain one or more natural or unnatural amino acid residues which are connected by peptide (amide) linkages. Different types of linkers may also be combined to new linkers. The different oligonucleotides are synthesized by established methods and can be linked together on-line during solid-phase synthesis. Alternatively, they may be linked together post-synthesis of the individual partial sequences.

In some embodiments of the invention the TLR ligand and anti-viral agent are linked such that they are part of the same molecule. TLR ligands can be linked to anti-viral agents directly or via non-nucleotidic linkers. A TLR ligand is “linked directly” if it is covalently bound to the oligonucleotide with no intervening structures. An oligonucleotide is said to be “linked indirectly” if it is connected to the oligonucleotide via a linker.

The linker connecting the oligonucleotide and anti-viral agent may contain a nuclease susceptible site. A “nuclease susceptible site” as used herein refers to a DNA or RNA sequence that is recognized and cleaved by a member of the class of enzymes known as nucleases. In some embodiments, the nuclease susceptible site is recognized and cleaved by a nuclease naturally present in the target cell.

In some embodiments the anti-viral agent or the linker is conjugated to an internal nucleotide of the immunostimulatory oligonucleotide. An “internal nucleotide” as used herein refers to a nucleotide that is not at the extreme 3′ or 5′ terminus of the nucleic acid polymer. A “terminal nucleotide”, therefore, refers to a nucleotide at either the 3′ or 5′ terminus of the nucleic acid polymer. In some embodiments the anti-viral agent or the linker is conjugated to a terminal nucleotide. As used herein, the “3′ terminal nucleotide” refers to the nucleotide residue at the extreme 3′ terminus of the oligonucleotide polymer. Similarly, the “5′ terminal nucleotide” refers to the nucleotide residue at the extreme 5′ terminus of the oligonucleotide polymer. In some embodiments the immunostimulatory oligonucleotide may comprise an internal 3′-3′ linkage or 5′-5′ linkage. In such cases, the immunostimulatory oligonucleotide with have two 5′ or 3′ linkages, respectively. If the anti-viral agent is a nucleotide or oligonucleotide, the anti-viral agent can also be conjugated to the immunostimulatory oligonucleotide through a 3′-5′, 3′-3′ or 5′-5′ linkage.

In some aspects of the invention the TLR ligand and the anti-viral agent are not linked but are administered together in the context of a microparticle. A “microparticle” as used herein is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”, herein incorporated by reference. PCT/US/0307 describes a biocompatible, preferably biodegradable polymeric matrix for containing an exogenous gene under the control of an appropriate promoter. The polymeric matrix can be used to achieve sustained release of the exogenous gene in the patient.

The polymeric matrix preferably is in the form of a microparticle such as a microsphere (wherein the immunostimulatory oligonucleotide and anti-viral agent or agents are dispersed throughout a solid polymeric matrix) or a microcapsule (wherein the immunostimulatory oligonucleotide and anti-viral agent or agents are stored in the core of a polymeric shell). Other forms of the polymeric matrix for containing the immunostimulatory oligonucleotide and anti-viral agent or agents include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid, antiviral agent, and/or allergen are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

Both non-biodegradable and biodegradable polymeric matrices can be used to deliver the TLR ligand and/or antiviral to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

As used herein, the term “treat” as used in reference to a subject having a disease or condition shall mean to prevent, ameliorate, or eliminate at least one sign or symptom of the disease or condition in the subject.

The compositions described herein may be used in the treatment of cancer.

A subject having a cancer is a subject that has detectable cancerous cells. The cancer may be a malignant or non-malignant cancer. “Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location, resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and central nervous system (CNS) cancer; breast cancer; cervical cancer; choriocarcinoma; colon and rectum cancer; connective tissue cancer; cancer of the digestive system; endometrial cancer; esophageal cancer; eye cancer; cancer of the head and neck; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, as well as other carcinomas, adenocarcinomas, and sarcomas.

The immunostimulatory composition of the invention may also be administered in conjunction with an anti-cancer therapy. Anti-cancer therapies include cancer medicaments, radiation, and surgical procedures. As used herein, a “cancer medicament” refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32Nalrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARD inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate, but it is not so limited.

The immunotherapeutic agent may be selected from the group consisting of 3622W94, 4B5, ANA Ab, anti-FLK-2, anti-VEGF, ATRAGEN, AVASTIN (bevacizumab; Genentech), BABS, BEC2, BEXXAR (tositumomab; GlaxoSmithKline), C225, CAMPATH (alemtuzumab; Genzyme Corp.), CEACIDE, CMA 676, EMD-72000, ERBITUX (cetuximab; ImClone Systems, Inc.), Gliomab-H, GNI-250, HERCEPTIN (trastuzumab; Genentech), IDEC-Y2B8, ImmuRAIT-CEA, ior c5, ior egf.r3, ior t6, LDP-03, LymphoCide, MDX-11, MDX-22, MDX-210, MDX-220, MDX-260, MDX-447, MELIMMUNE-1, MELIMMUNE-2, Monopharm-C, NovoMAb-G2, Oncolym, OV103, Ovarex, Panorex, Pretarget, Quadramet, Ributaxin, RITUXAN (rituximab; Genentech), SMART 1D10 Ab, SMART ABL 364 Ab SMART M195, TNT, and ZENAPAX (daclizumab; Roche), but it is not so limited.

The invention also involves methods of treating bacterial infections. A “subject having an infection” is a subject that has a disorder arising from the invasion of the subject, superficially, locally, or systemically, by an infectious microorganism. The infectious microorganism can be a virus or bacterium.

Bacteria are unicellular organisms which multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

Infectious bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria sps (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidum, Treponema pertenue, Leptospira, Rickettsia, and Actinomyces israelli.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative and is not intended to be limiting.

The methods of the invention can further include the administration of anti-bacterial agents. Anti-bacterial agents kill or inhibit bacteria, and include antibiotics as well as other synthetic or natural compounds having similar functions. Many antibiotics are low molecular weight molecules which are produced as secondary metabolites by cells, such as microorganisms. In general, antibiotics interfere with one or more functions or structures which are specific for the microorganism and which are not present in host cells.

Antibacterial antibiotics which are effective for killing or inhibiting a wide range of bacteria are referred to as broad-spectrum antibiotics. Other types of antibacterial antibiotics are predominantly effective against the bacteria of the class gram-positive or gram-negative. These types of antibiotics are referred to as narrow-spectrum antibiotics. Other antibiotics which are effective against a single organism or disease and not against other types of bacteria, are referred to as limited-spectrum antibiotics.

Anti-bacterial agents are sometimes classified based on their primary mode of action. In general, anti-bacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The compounds of the invention may be administered alone (e.g. in saline or buffer) or using any delivery vectors known in the art. The TLR ligands and antiviral agents can be combined with other therapeutic agents such as adjuvants to enhance immune responses even further. The TLR ligand and/or antiviral agent and/or other therapeutic agent may be administered simultaneously or sequentially. When the other therapeutic agents are administered simultaneously they can be administered in the same or separate formulations, but are administered at the same time. The other therapeutic agents are administered sequentially with one another and with the TLR ligand and antiviral agent, when the administration of the other therapeutic agents and the TLR ligand and antiviral agent is temporally separated. The separation in time between the administration of these compounds may be a matter of minutes or it may be longer. Other therapeutic agents include but are not limited to non-nucleic acid adjuvants, cytokines, antibodies, antigens, etc.

A non-nucleic acid adjuvant is any molecule or compound except for the immunostimulatory nucleic acids described herein which can stimulate the humoral and/or cellular immune response. Non-nucleic acid adjuvants include, for instance, adjuvants that create a depo effect, immune stimulating adjuvants, adjuvants that create a depo effect and stimulate the immune system and mucosal adjuvants.

An adjuvant that creates a depo effect as used herein is an adjuvant that causes an antigen to be slowly released in the body, thus prolonging the exposure of immune cells to the antigen. An immune stimulating adjuvant is an adjuvant that causes activation of a cell of the immune system. “Adjuvants that create a depo effect and stimulate the immune system” are those compounds which have both of the above-identified functions. A “non-nucleic acid mucosal adjuvant” as used herein is an adjuvant other than an immunostimulatory nucleic acid that is capable of inducing a mucosal immune response in a subject when administered to a mucosal surface in conjunction with an antigen. Such molecules are described for instance, in U.S. patent application Ser. No. 10/888,886 published as US 2004/0266719 and U.S. Pat. No. 6,406,705 each of which are incorporated by reference.

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the immunostimulatory nucleic acids and antiviral agents. The cytokines can be administered directly with immunostimulatory nucleic acids or may be administered in the form of a nucleic acid vector that encodes the cytokine, such that the cytokine can be expressed in vivo. In one embodiment, the cytokine is administered in the form of a plasmid expression vector. In this embodiment, the immunostimulatory nucleic acid is not contained within the same plasmid. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, IL-18 granulocyte-macrophage colony stimulating factor (GM-CSF), granulocyte colony stimulating factor (GCSF), interferon-γ (γ-IFN), IFN-α, tumor necrosis factor (TNF), TGF-β, FLT-3 ligand, and CD40 ligand. Cytokines play a role in directing the T cell response. Helper (CD4+) T cells orchestrate the immune response of mammals through production of soluble factors that act on other immune system cells, including other T cells. Most mature CD4+T helper cells express one of two cytokine profiles: Th1 or Th2. In some embodiments it is preferred that the cytokine be a Th1 cytokine.

The term “effective amount” of a TLR ligand and an antiviral agent refers to the amount necessary or sufficient to realize a desired biologic effect. For example, an effective amount of an immunostimulatory nucleic acid and an antiviral agent for treating or preventing infectious disease is that amount necessary to prevent the infection with the microorganism if the subject is not yet infected or is that amount necessary to prevent an increase in infected cells or microorganisms present in the subject or that amount necessary to decrease the amount of the infection that would otherwise occur in the absence of the immunostimulatory nucleic acid or antiviral agent when either is used alone. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular immunostimulatory nucleic acid or antiviral agent being administered (e.g. the type of nucleic acid, i.e. a CpG nucleic acid, the number of immunostimulatory motifs or their location in the nucleic acid, the degree of modification of the backbone to the oligonucleotide the type of medicament), the size of the subject, or the severity of the disease or condition. One of ordinary skill in the art can empirically determine the effective amount of a particular immunostimulatory nucleic acid and/or antiviral agent and/or other therapeutic agent without necessitating undue experimentation.

In some embodiments of the invention, the TLR ligand and antiviral agent are administered in a synergistic amount effective to treat or prevent infectious disease. A synergistic amount is that amount which produces a physiological response that is greater than the sum of the individual effects of either the immunostimulatory nucleic acid or the antiviral agent alone. For instance, in some embodiments of the invention, the physiological effect is a reduction in the number of cells infected with the virus. A synergistic amount is that amount which produces a reduction in infected cells that is greater than the sum of the infected cells reduced by either the immunostimulatory nucleic acid or the antiviral agent alone. In other embodiments, the physiological result is a reduction in the number of microorganisms in the body. The synergistic amount in this case is that amount which produces the reduction that is greater than the sum of the reduction produced by either the immunostimulatory nucleic acid or the antiviral agent alone. In other embodiments the physiological result is a decrease in physiological parameters associated with the infection, e.g., fungal lesions or other symptoms. For instance, a diagnosis of urinary tract infection is based on the presence and quantification of bacteria in the urine when greater than 10⁵ colonies per milliliter of microorganisms are detected in a mid-stream, clean-voided urine specimen. A reduction in this number to 10³ and preferably to fewer than 10² bacterial colonies per milliliter indicates that the infection has been eradicated.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

In some instances, a sub-therapeutic dosage of the TLR ligand and the antiviral agent are used. When the two classes of drugs are used together, they can be administered in sub-therapeutic doses and still produce a desirable therapeutic result, a “sub-therapeutic dose” as used herein refers to a dosage which is less than that dosage which would produce a therapeutic result in the subject. Thus, the sub-therapeutic dose of an antiviral agent is one which would not produce the desired therapeutic result in the subject in the absence of the immunostimulatory nucleic acid. Therapeutic doses of antiviral agent are well known in the field of medicine for the treatment of infectious disease. These dosages have been extensively described in references such as Remington's Pharmaceutical Sciences, 18th ed., 1990; as well as many other medical references relied upon by the medical profession as guidance for the treatment of infectious disease. Therapeutic dosages of immunostimulatory oligonucleotides have also been described in the art and methods for identifying therapeutic dosages in subjects are described in more detail above.

In other embodiments of the invention, the TLR ligand and antiviral agent are administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the composition on a daily basis for the first week, followed by a monthly basis for several months, and then every three months after that. Any particular combination would be covered by the routine schedule as long as it is determined ahead of time that the appropriate schedule involves administration on a certain day.

For any compound described herein the therapeutically effective amount can be initially determined from animal models. A therapeutically effective dose can also be determined from human data for CpG oligonucleotides which have been tested in humans (human clinical trials have been initiated) and for compounds which are known to exhibit similar pharmacological activities, such as other adjuvants, e.g., LT and other antigens for vaccination purposes. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the TLR ligand and anti viral composition can be administered to a subject by any mode that delivers the composition to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the oligonucleotide (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the oligonucleotide (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the oligonucleotide or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the oligonucleotides (or derivatives thereof). The oligonucleotide (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of oligonucleotide (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified oligonucleotide may also be prepared in different formulations depending on the type of chemical modification or the type of device employed.

Formulations suitable for use with a nebulizer, either jet or ultrasonic, will typically comprise oligonucleotide (or derivative) dissolved in water at a concentration of about 0.1 to 25 mg of biologically active oligonucleotide per mL of solution. The formulation may also include a buffer and a simple sugar (e.g., for oligonucleotide stabilization and regulation of osmotic pressure). The nebulizer formulation may also contain a surfactant, to reduce or prevent surface induced aggregation of the oligonucleotide caused by atomization of the solution in forming the aerosol.

Formulations for use with a metered-dose inhaler device will generally comprise a finely divided powder containing the oligonucleotide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided thy powder containing oligonucleotide (or derivative) and may also include a bulking agent, such as lactose, sorbitol, sucrose, or mannitol in amounts which facilitate dispersal of the powder from the device, e.g., 50 to 90% by weight of the formulation. The oligonucleotide (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a composition optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

In other aspects, the invention relates to kits that are useful in the treatment of infectious disease. One kit of the invention includes a container housing an immunostimulatory nucleic acid and a container housing an antiviral agent and instructions for timing of administration of the immunostimulatory nucleic acid and the antiviral agent. Preferably, the immunostimulatory nucleic acid is provided for systemic administration, and the instructions accordingly provide for this. In an important embodiment, the container housing the immunostimulatory nucleic acid is a sustained release vehicle is used herein in accordance with its prior art meaning of any device which slowly releases the immunostimulatory nucleic acid.

In addition to the use of the TLR ligands and anti-viral agents to prevent infection in humans, the compositions are also suited for treatment of non-human vertebrates. Non-human vertebrates which exist in close quarters and which are allowed to intermingle as in the case of zoo, farm and research animals are also embraced as subjects for the methods of the invention. Zoo animals such as the felid species including for example lions, tigers, leopards, cheetahs, and cougars; elephants, giraffes, bears, deer, wolves, yaks, non-human primates, seals, dolphins and whales; and research animals such as mice, rats, hamsters and gerbils are all potential subjects for the methods of the invention.

Birds such as hens, chickens, turkeys, ducks, geese, quail, and pheasant are prime targets for many types of infections. Hatching birds are exposed to pathogenic microorganisms shortly after birth. Although these birds are initially protected against pathogens by maternal derived antibodies, this protection is only temporary, and the bird's own immature immune system must begin to protect the bird against the pathogens. It is often desirable to prevent infection in young birds when they are most susceptible. It is also desirable to prevent against infection in older birds, especially when the birds are housed in closed quarters, leading to the rapid spread of disease. Thus, it is desirable to administer the immunostimulatory oligonucleotides and anti-viral agents to birds to prevent infectious disease.

An example of a common infection in chickens is chicken infectious anemia virus (CIAV). CIAV was first isolated in Japan in 1979 during an investigation of a Marek's disease vaccination break (Yuasa et al., 1979, Avian Dis. 23:366-385). Since that time, CIAV has been detected in commercial poultry in all major poultry producing countries (van Bulow et al., 1991, pp. 690-699) in Diseases of Poultry, 9th edition, Iowa State University Press).

CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fungal infections which then complicate the course of the disease. The immunosuppression may cause aggravated disease after infection with one or more of Marek's disease virus (MDV), infectious bursal disease virus, reticuloendotheliosis virus, adenovirus, or reovirus. It has been reported that pathogenesis of MDV is enhanced by CIAV (DeBoer et al., 1989, p. 28 In Proceedings of the 38th Western Poultry Diseases Conference, Tempe, Ariz.). Further, it has been reported that CIAV aggravates the signs of infectious bursal disease (Rosenberger et al., 1989, Avian Dis. 33:707-713). Chickens develop an age resistance to experimentally induced disease due to CAA. This is essentially complete by the age of 2 weeks, but older birds are still susceptible to infection (Yuasa, N. et al., 1979 supra; Yuasa, N. et al., Arian Diseases 24, 202-209, 1980). However, if chickens are dually infected with CAA and an immunosuppressive agent (IBDV, MDV etc.) age resistance against the disease is delayed (Yuasa, N. et al., 1979 and 1980 supra; Bulow von V. et al., J. Veterinary Medicine 33, 93-116, 1986). Characteristics of CIAV that may potentiate disease transmission include high resistance to environmental inactivation and some common disinfectants. The economic impact of CIAV infection on the poultry industry is clear from the fact that 10% to 30% of infected birds in disease outbreaks die.

Cattle and livestock are also susceptible to infection. Diseases which affect these animals can result in severe economic losses, especially amongst cattle. The methods of the invention can be used to protect against infection in livestock, such as cows, horses, pigs, sheep, and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (HCV) groups (Francki, et al., 1991).

BVDV, which is an important pathogen of cattle can be distinguished, based on cell culture analysis, into cytopathogenic (CP) and noncytopathogenic (NCP) biotypes. The NCP biotype is more widespread although both biotypes can be found in cattle. If a pregnant cow becomes infected with an NCP strain, the cow can give birth to a persistently infected and specifically immunotolerant calf that will spread virus during its lifetime. The persistently infected cattle can succumb to mucosal disease and both biotypes can then be isolated from the animal. Clinical manifestations can include abortion, teratogenesis, and respiratory problems, mucosal disease and mild diarrhea. In addition, severe thrombocytopenia, associated with herd epidemics, that may result in the death of the animal has been described and strains associated with this disease seem more virulent than the classical BVDVs.

Equine herpesviruses (EHV) comprise a group of antigenically distinct biological agents which cause a variety of infections in horses ranging from subclinical to fatal disease. These include Equine herpesvirus-1 (EHV-1), a ubiquitous pathogen in horses. EHV-1 is associated with epidemics of abortion, respiratory tract disease, and central nervous system disorders. Primary infection of upper respiratory tract of young horses results in a febrile illness which lasts for 8 to 10 days. Immunologically experienced mares may be reinfected via the respiratory tract without disease becoming apparent, so that abortion usually occurs without warning. The neurological syndrome is associated with respiratory disease or abortion and can affect animals of either sex at any age, leading to in-coordination, weakness and posterior paralysis (Telford, E. A. R. et al., Virology 189, 304-316, 1992). Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus. Inactivated cell-virus and cell-free whole simian immunodeficiency vaccines have been reported to afford protection in macaques (Stott et al. (1990) Lancet 36:1538-1541; Desrosiers et al. PNAS USA (1989) 86:6353-6357; Murphey-Corb et al. (1989) Science 246:1293-1297; and Carlson et al. (1990) AIDS Res. Human Retroviruses 6:1239-1246). A recombinant HIV gp120 vaccine has been reported to afford protection in chimpanzees (Berman et al. (1990) Nature 345:622-625).

Cats, both domestic and wild, are susceptible to infection with a variety of microorganisms. For instance, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to prevent or treat infection in cats.

Domestic cats may become infected with several retroviruses, including but not limited to feline leukemia virus (FeLV), feline sarcoma virus (FeSV), endogenous type C oncornavirus (RD-114), and feline syncytia-forming virus (FeSFV). Of these, FeLV is the most significant pathogen, causing diverse symptoms, including lymphoreticular and myeloid neoplasms, anemias, immune mediated disorders, and an immunodeficiency syndrome which is similar to human acquired immune deficiency syndrome (AIDS). Recently, a particular replication-defective FeLV mutant, designated FeLV-AIDS, has been more particularly associated with immunosuppressive properties.

The discovery of feline T-lymphotropic lentivirus (also referred to as feline immunodeficiency) was first reported in Pedersen et al. (1987) Science 235:790-793. Characteristics of FIV have been reported in Yamamoto et al. (1988) Leukemia, December Supplement 2:204 S-215S; Yamamoto et al. (1988) Am. J. Vet. Res. 49:1246-1258; and Ackley et al. (1990) J. Virol. 64:5652-5655. Cloning and sequence analysis of FIV have been reported in Olmsted et al. (1989) Proc. Natl. Acad. Sci. USA 86:2448-2452 and 86:4355-4360.

Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat, and pallas cat. In domestic cats, the disease occurs predominantly in young animals, although cats of all ages are susceptible. A peak incidence occurs between 6 and 12 months of age. A decline in incidence is noted from 5 to 13 years of age, followed by an increased incidence in cats 14 to 15 years old.

The invention further encompasses a method of screening for molecules containing an anti-viral agent and an immunostimulatory oligonucleotide for immune stimulatory activity and, simultaneously, for anti-viral activity. This may be achieved either by using immune cells isolated from virus-infected patients, for example by measuring cytokine production and virus titer, or a combination of an in vitro anti-viral test system, for example the HCV replicon and an in vivo immune stimulatory test system, for example a cell line bearing the TLR9/IFN-alpha signaling pathway, or an in vitro viral test system mimicking a viral infection (e.g., bovine viral diarrhea virus infected PBMC for HCV). In addition, as many viruses target with their proteins anti-viral effects, such as TLR-mediated signaling, cell systems either naturally containing or transfected with the TLR signaling pathway of interest and the anti-viral agent may be used to screen for the combined effect of an anti-viral and an immunostimulatory oligonucleotide.

The screening methods of the invention are useful for identification of effective anti-viral compositions of immunostimulatory oligonucleotides and anti-viral therapy. One screening method employs isolation of immune cells from virus-infected patients followed by treatment of the cells with the compositions of the invention. The effectiveness of the compositions may be evaluated by measuring cytokine production and virus titer. Such measurements may be done by using vitro anti-viral test system, for example the HCV replicon (reference needed) and an in vivo immune stimulatory test system, for example a cell line bearing the TLR9/IFN-alpha signaling pathway. Another in vitro viral test system that may be used is one that mimics a viral infection, such as PBMC infected with bovine viral diarrhea virus as a model for HCV. In addition, as many viruses target anti-viral processes in the body, such as TLR-mediated signaling, cell systems either naturally containing or transfected with the TLR signaling pathway of interest and the anti-viral composition may be used to screen for the combined effect of an anti-viral and an immunostimulatory oligonucleotide. For example, in one such combination the anti-viral agent is the NS3/4 protease, and the immunostimulatory oligonucleotide is a CpG oligonucleotide.

### EXAMPLES

### Methods

Oligonucleotides and reagents All ODN and ORN were purchased from Biospring (Frankfurt, Germany) or provided by Coley Pharmaceutical GmbH (Langenfeld, Germany), controlled for identity and purity by Coley Pharmaceutical GmbH and had undetectable endotoxin levels (<0.1EU/ml) measured by the Limulus assay (BioWhittaker, Verviers, Belgium). ODN were suspended in sterile, endotoxin-free Tris-EDTA (Sigma, Deisenhofen, Germany), ORN were suspended in sterile, DNAse- and RNAse-free dH₂O (Life Technologies, Eggenstein, Germany) and stored and handled under aseptic conditions to prevent both microbial and endotoxin contamination. All dilutions were carried out using endotoxin-free Tris-EDTA or DNAse- and RNAse-free dH₂O, Nucleosides including 8-Oxo-rG and chloroquine were obtained from Sigma or ChemGenes (Wilmington, Mass., USA), and were dissolved in DMSO, NaOH or H₂O.

Cell purification Peripheral blood buffy coat preparations from healthy human donors were obtained from the Blood Bank of the University of Düsseldorf (Germany) and PBMC were purified by centrifugation over Ficoll-Hypaque (Sigma). Cells were cultured in a humidified incubator at 37° C. in RPMI 1640 medium supplemented with 5% (v/v) heat inactivated human AB serum (BioWhittaker) or 10% (v/v) heat inactivated FCS, 2 mM L-glutamine, 100 U/ml penicillin and 100 μg/ml streptomycin (all from Sigma).

Cytokine detection PBMC were resuspended at a concentration of 5×10⁶ cells/ml and added to 96 well round-bottomed plates (250 μl/well). PBMC were incubated with various ODN, ORN or nucleoside concentrations and culture supernatants (SN) were collected after the indicated time points. If not used immediately, SN were stored at −20° C. until required. For inhibitory experiments, cells were stimulated with the indicated TLR ligand concentration and nucleoside or ORN added. In some experiments, the second modified ORN was added 1 h after the start of the cell culture.

Amounts of cytokines in the SN were assessed using a commercially available ELISA Kit for IL-12p40 (from BD Biosciences, Heidelberg, Germany), IFN-γ and TNF-α (from Diaclone, Besancon, France) or an in-house ELISA for IFN-α developed using commercially available antibodies (PBL, New Brunswick, N.J., USA). For analysis of a broad set of cytokines and chemokines, multiplex analysis with a luminex system from Bio-Rad (Munich, Germany) and Multiplex kits from Biosource (Solingen, Germany) was performed.

### Examples

### Example 1

### Synergistic Effect of Linking an 8-Modified Guanosine to an Immune Stimulatory Nucleic Acid was Observed

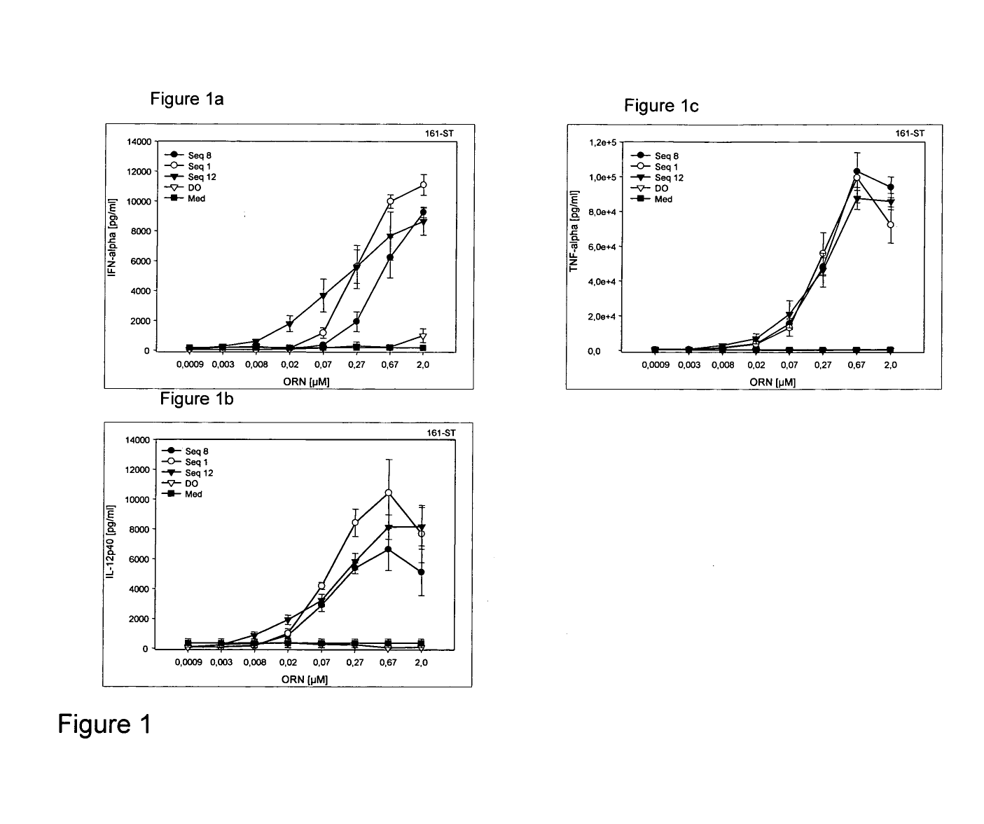

Human PBMC (n=3) were stimulated with 8-Oxo-rG (a C-8 substituted guanosine) modified ORN (SEQ ID NO:1-4) and an unmodified ORN (SEQ ID NO:8). Supernatants were collected and cytokines IFN-alpha (FIG. 1a), IL-12p40 (FIG. 1b) and TNF-alpha (FIG. 1c) were measured. The data demonstrate that the addition of 8-Oxo-rG in the sequence enhances the IFN-alpha and IL-12 inducing activity when compared to the unmodified ORN SEQ ID NO:10.

### Example 2

### The Position of 8-Modified G in the RNA Sequence May Enhance Activity

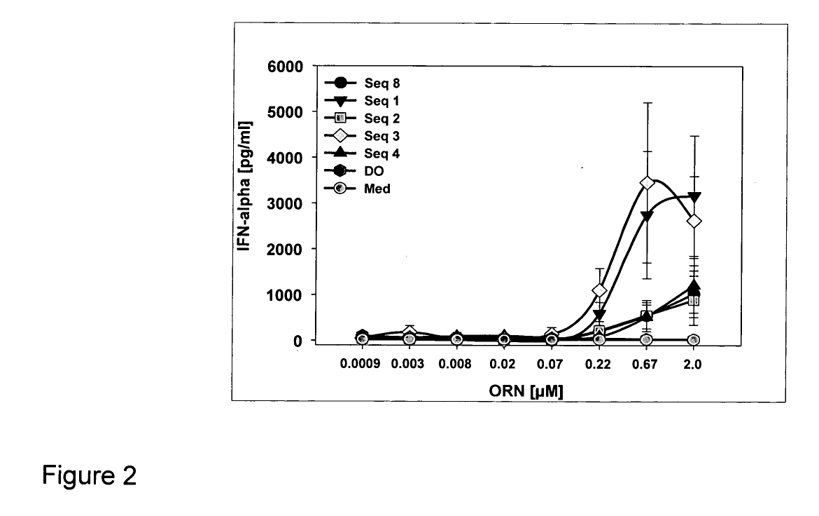

Human PBMC (n=3) were stimulated with the indicated ORN with a single 8-Oxo-rG at different positions of the ORN (SEQ ID NO:1-4) and the 8-Bromo-dA modified negative control (SEQ ID NO:8). Supernatants were collected and IFN-alpha measured. The data show that the inclusion of an 8-Oxo-rG at the 5′ position (SEQ ID NO:1 and 3), but not at positions further 3′ (SEQ ID NO:2 and 4), results in the increased cytokine induction (FIG. 2).

### Example 3

### Different 8-Modified Deoxy- and Ribonucleotides at the ORN 5′ End Increase the Immune Stimulatory Activity

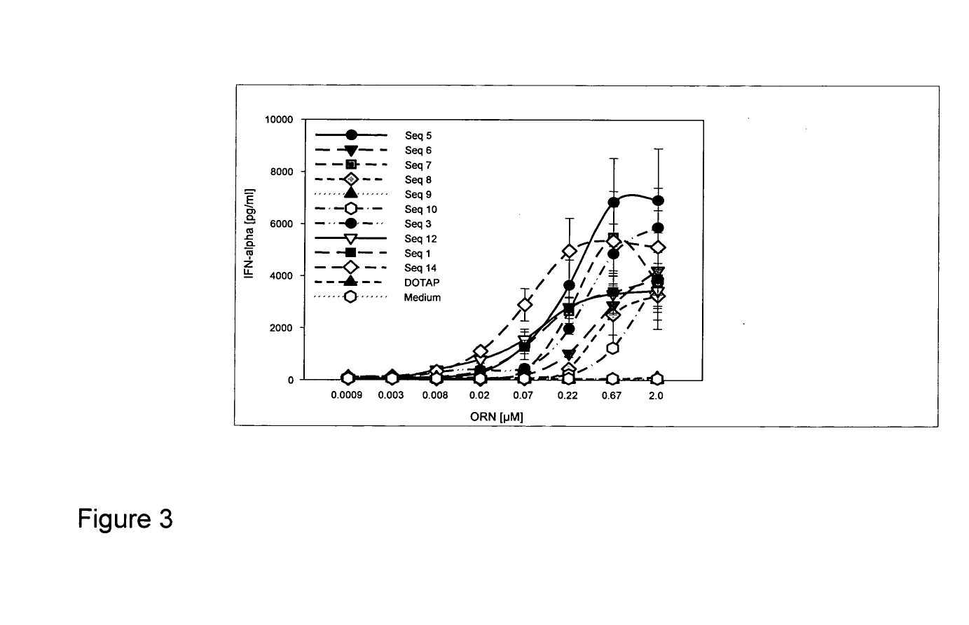

Human PBMC (n=3) were stimulated with the indicated ORN with a single 8-Oxo-rG/Dg (SEQ ID NO:1, 5), 8-Bromo-dG (SEQ ID NO:7) or Immunosine (Isatoribine) (SEQ ID NO:6) (with a 5′-5′ linkage) at the 5′ end of the ORN and IFN-alpha measured. The data show that the addition of 8-modified Gs (either deoxy- or ribonucleotides) at the 5′ position resulted in the increased cytokine induction compared to the unmodified ORN (SEQ ID NO:8 and 11) (FIG. 3). Similar data were obtained for 8-Bromo-rG (data not shown). In addition, even the linkage of a 8-modified dA or rA (data not shown) results in an, although only slight increase of the cytokine induction.

### Example 4

### The Combination of Ribavirin with an Immune Stimulatory CpG ODN Results in a Decrease of the IL-10 Relative to the IFN-alpha Inducing Activity

Upon stimulation of human PBMC with a CpG ODN (SEQ ID NO:15) and co-culture with increasing doses of Ribavirin, a suppressive effect of the Ribavirin on the CpG-induced IL10 induction can be observed. Importantly, the suppressive effect of Ribavirin was not observed with respect to IFN-alpha. The observations were particularly noted at lose doses of Ribavirin.

TABLE 1
calculated IC50 upon addition of Ribavirin
to cultures containing 1 μM SEQ ID NO: 13

| | IC50 IFN-alpha | IC50 IL-10 | IC50 IL-6 |
|---|---|---|---|
| Ribavirin | 1000 μM | 58 μM | 320 μM |

The observation has important implications for the use of Ribavirin in therapeutic indications. IL-10 is a regulatory cytokine that often counteracts therapeutic interventions (e.g., for TLR ligands, or IFN-alpha therapy). The ability of suppress IL10 is useful in drug combinations because it enables IFN and other cytokines to produce an enhanced response, thus increasing the efficacy of the combination of therapeutic agents. Therefore, this effect of Ribavirin may result in an alteration of the cytokine milieu in the patient treated with a combination of the above and Ribavirin, resulting in a stronger, uninhibited effect of the cytokine or TLR ligand.

TABLE 2

Nucleic acid sequences

| | |
|---|---|
| Seq ID No. | Sequence |

| | |
|---|---|
| 1 | rO*rU*rU*rO*rU*rO*rU |
| 2 | rG*rU*rU*rO*rU*rG*rU |
| 3 | rO*rU*rU*rG*rU*rG*rU |
| 4 | rG*rU*rU*rG*rU*rO*rU |
| 5 | O*rU*rU*rG*rU*rG*rU |
| 6 | iIM*rU*rU*rG*rU*rG*rU |
| 7 | BG*rU*rU*rG*rU*rG*rU |
| 8 | rG*rU*rU*rG*rU*rG*rU |
| 9 | rG*rC*rC*rA*rC*rC*rG*rA*rG*rC*rC*rG*rA*rA*rG*rG*rC*rA*rC*rC |
| 10 | BA*rU*rU*rG*rU*rG*rU |
| 11 | rC*rC*rG*rU*rC*rU*rG*rU*rU*rG*rU*rG*rU*rG*rA*rC*rU*rC |
| 12 | rU*rU*rG*rU*rU*rG*rU*rU*rG*rU*rU*rG*rU*rU*rG*rU*rU*rG*rU*rU |
| 13 | T*C*G*T*C*G*T*T*T*T*C*G*G*C*G*C*G*C*G*C*C*G |
| 14 | TCCATGACGTTCCTGATGC |

### Example 5

### Effect of Ribavirin In Vitro and In Vivo on an Immune Stimulatory CpG ODN Mediated T Cell Cytokine Production

Initial experiment with 10 mg/kg intraperitoneal (IP) ribivirin (RBV) showed no effect in vivo of RBV and possibly even a slight decrease in ex vivo IFN-γ CD3-induced production. However, ex vivo anti-CD3 stimulation in the presence of RBV showed increase IFN-γ production, but only at rather low concentrations of RBV (1-5 μM). We repeated the experiment with a lower dose of RBV in vivo (0.5 mg/kg IP).

Mice were injected with SEQ ID NO. 14 (100 micrograms SC), RBV (0.5 mg/kg IP) or SEQ ID NO. 14 and RBV. RBV was administered in vivo either at day 0 or day 5 and CpG was administered at day O, Samples were isolated at Day 6 from the inguinal lymph node and maintained in medium on anti-CD3 coated plated in the presence of 1-16 μM RBV. On Day 8 an ELISA assay was performed to detect IFN-gamma.

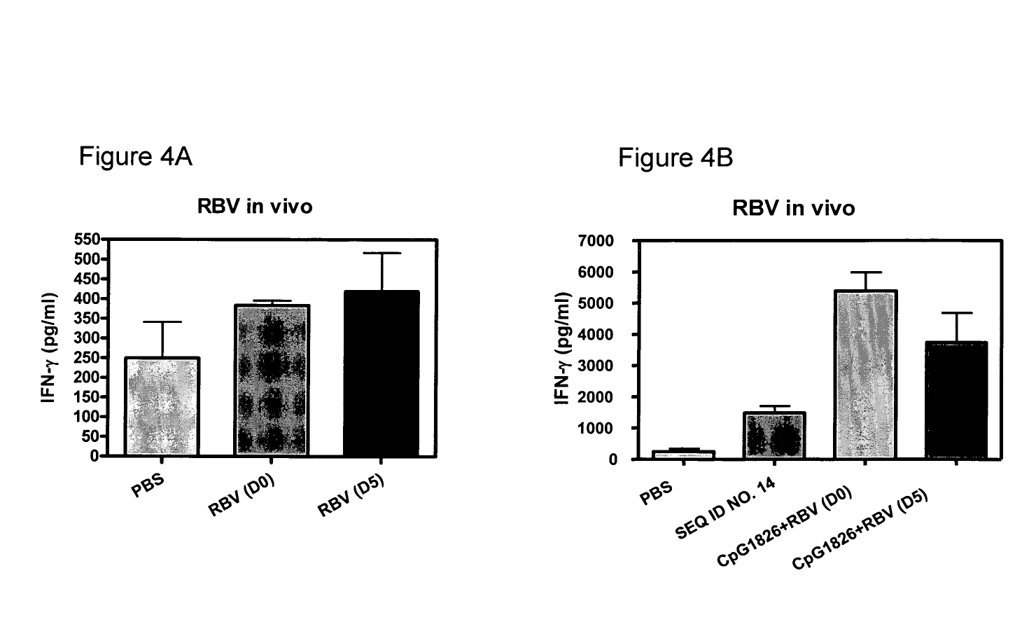

As expected, in vivo CpG ODN increased T cell IFN-γ production ex-vivo (FIG. 4B). A small effect of RBV on IFN-γ production in the absence of CpG ODN was observed (FIG. 4A). However, the effect of RBV on IFN-γ production was greatly enhanced when CpG ODN was also injected.

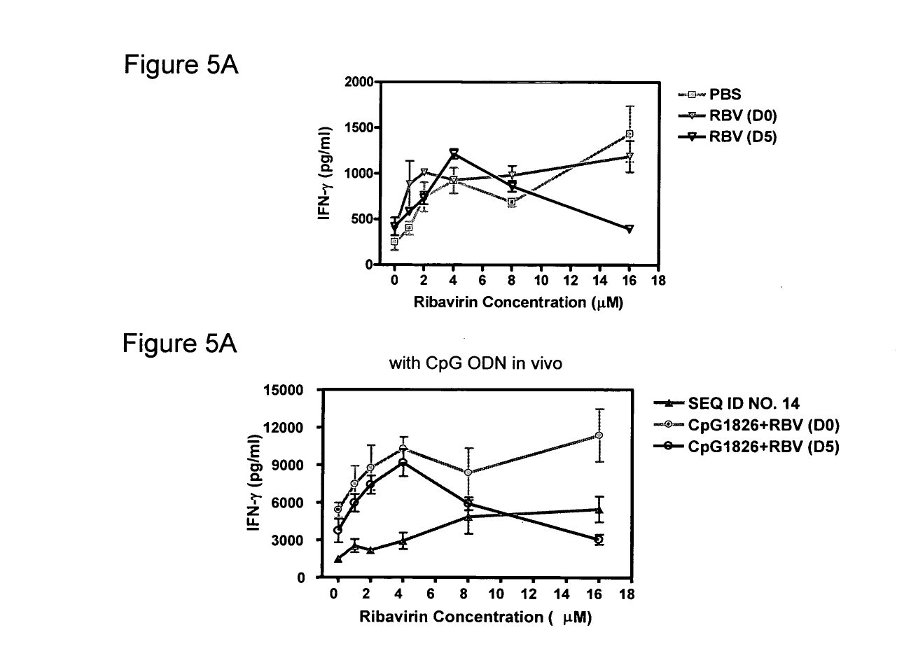

The ex vivo effect of RBV on CD3-mediated IFN-γ production independently of prior ODN/RBV treatment was examined. The results are shown in FIG. 5. Similar to the data described above, low concentrations of RBV in vitro increased IFN-γ levels independently of previous in vivo treatments (FIG. 5A). The effect of a combination with CpG ODN is shown in FIG. 5B.

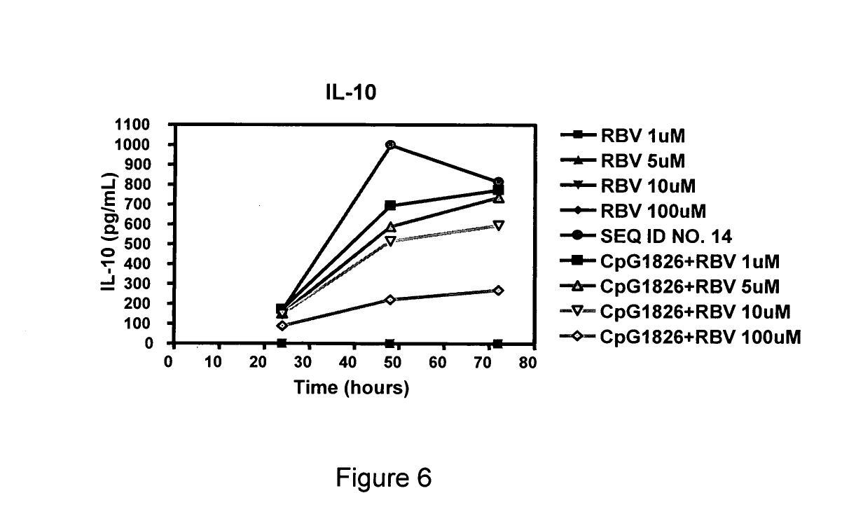

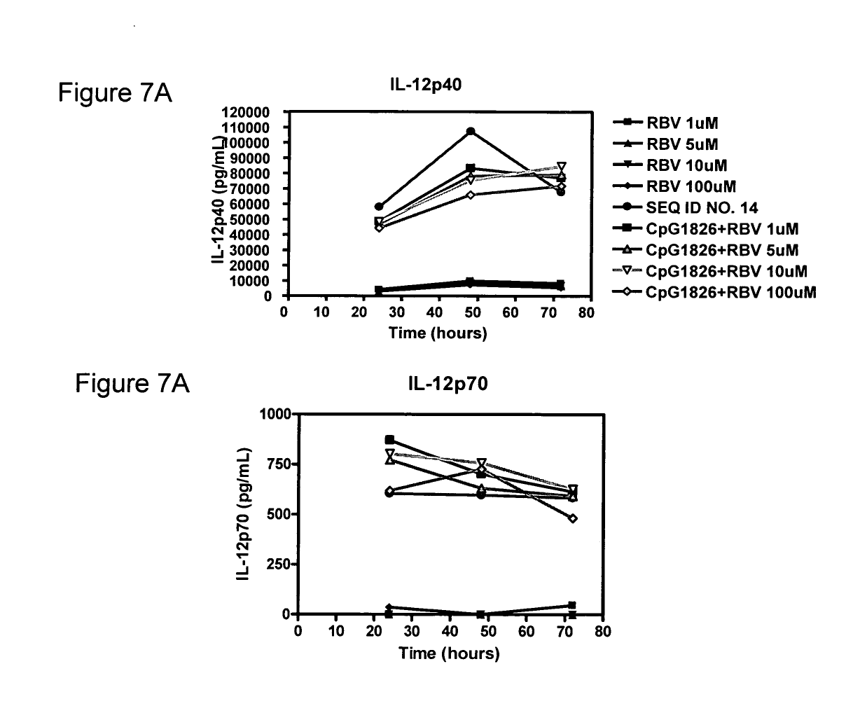

Similar experiments were performed using bone marrow (BM) derived dendritic cells (DCs). BM-derived DC maintained in GM-CSF were treated with SEQ ID NO. 14, RBV (1 μM, 5 μM, 10 μM, 100 μM, or 120 μM) or with SEQ ID NO. 14 and RBV. At 4, 24 and 72 hours samples were tested for cytokine, IL-10 (FIG. 6), IL-12p40 (FIG. 7A), IL-12p70 (FIG. 7B) and TNF (data not shown). No effects of RBV alone at 120 μM were observed. SEQ ID NO. 14 induced IL-12, IL-10 and TNF. RBV decreased IL-10 and TNF but increased IL-12 (at 72 h only). RBV decreased SEQ ID NO. 14-induced IL-10 as shown in FIG. 6. RBV decreased SEQ ID NO. 14-induced IL-12p40 and increased IL-12p70 (slightly).

### Example 6

### Effect of Ribavirin and CpG ODN In Vivo in a Mouse Cancer Model

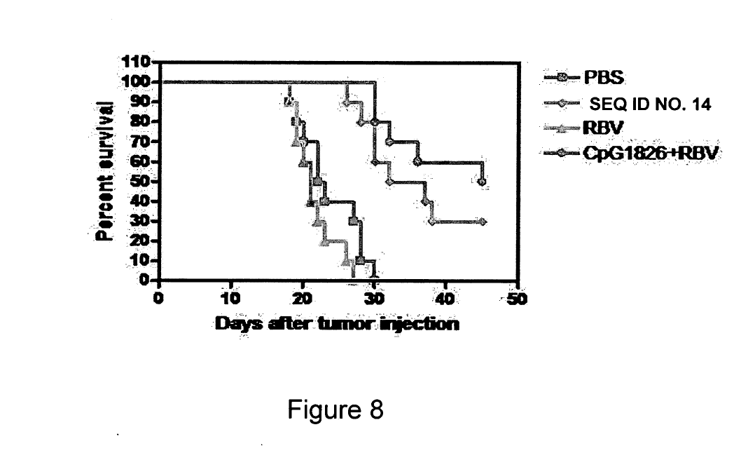

C26 SC mouse model was treated with SEQ ID NO. 14 and RBV (100 micrograms ODN intra/peri tumor at days 7, 14, 21 and 0.5 mg/kg RBV IP on same days). The data is shown in FIG. 8. As of days 30-40 the CpG ODN alone and the combined therapy produced prolonged survival in the mouse. Although the CpG ODN plus RBV did not achieve statistical significance by log-rank analysis over the CpG ODN therapy alone, there was a clear trend of improved survival, as shown in FIG. 8.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A composition comprising an immunostimulatory oligonucleotide and an anti-viral agent, wherein the anti-viral agent is not a C-8 substituted guanosine or an imidazoquinoline and wherein the anti-viral agent is linked to the immunostimulatory oligonucleotide.

2. The composition of claim 1, wherein the immunostimulatory oligonucleotide:
a) is linked to the anti-viral agent directly;
b) is linked to the anti-viral agent indirectly;
c) comprises a chimeric backbone;
d) an RNA oligonucleotide; or
e) a DNA oligonucleotide.

3. (canceled)

4. The composition of claim 2 a), wherein the immunostimulatory oligonucleotide and the anti-viral agent are part of the same molecule.

5. The composition of claim 1 wherein the anti-viral agent is one or more nucleotide analogues.

6. The composition of claim 2 a), further comprising a nuclease susceptible site between immunostimulatory oligonucleotide and the anti-viral agent.

7. The composition of claim 2 a), wherein the immunostimulatory oligonucleotide contains at least one 3′-3′ linkage or at least one 5′-5′ linkage.

8. (canceled)

9. The composition of claim 1, further comprising a pharmaceutically acceptable carrier.

10.-34. (canceled)

35. A composition of claim 2 d), wherein the anti-viral agent is associated with the immunostimulatory RNA oligonucleotide.

36. The composition of claim 35, wherein the immunostimulatory RNA oligonucleotide is:
a) linked to the anti-viral agent;
b) directly linked to the anti-viral agent; or
c) indirectly linked to the anti-viral agent.

37.-38. (canceled)

39. The composition of claim 36 a), wherein the immunostimulatory RNA oligonucleotide and the anti-viral agent are part of the same molecule.

40. The composition of claim 35, wherein the immunostimulatory RNA oligonucleotide is not linked to the anti-viral agent.

41. The composition of claim 40, wherein the composition includes
a) a microparticle housing the immunostimulatory RNA oligonucleotide and the anti-viral agent; or
b) a liposome housing the immunostimulatory RNA oligonucleotide and the anti-viral agent.

42. (canceled)

43. The composition of claim 35, wherein the anti-viral agent is:
a) one or more nucleotide analogues; or
b) a C-8 substituted guanosine.

44.-47. (canceled)

48. The composition of claim 43 b), wherein the C-8 substituted guanosine is incorporated in the RNA oligonucleotide.

49. The composition of claim 48, wherein the C-8 substituted guanosine is positioned at the 5′ end of the RNA oligonucleotide.

50. The composition of claim 48, wherein the C-8 substituted guanosine is positioned one, two or three nucleotides 3′ of the 5′ end of the RNA oligonucleotide.

51. A method for treating viral disease, comprising administering to a subject in need of such treatment a composition of claim 1 in an amount effective to treat the viral disease.

52.-73. (canceled)

74. A composition comprising a TLR7/8/9 ligand linked to an anti-viral agent.

75. The composition of claim 74, wherein the TLR7/8/9 ligand is an immunostimulatory oligonucleotide.

76. The composition of claim 74, wherein the TLR7/8/9 ligand is:
a) linked to the anti-viral agent directly; or
b) linked to the anti-viral agent indirectly.

77. (canceled)

78. The composition of claim 76 a), wherein the TLR7/8/9 ligand and the anti-viral agent are part of the same molecule.

79. The composition of claim 74 wherein the anti-viral agent is one or more nucleotide analogues.

80. The composition of claim 76 a), further comprising a nuclease susceptible site between the TLR7/8/9 ligand and the anti-viral agent.

81.-88. (canceled)

89. A method for treating bacterial infection comprising administering to a subject having a bacterial infection a composition of claim 1 in an amount effective to treat the bacterial infection.

90. The method of claim 89, wherein the anti-viral agent is linked to the immunostimulatory oligonucleotide.

91. (canceled)

92. The method of claim 89, wherein the immunostimulatory oligonucleotide is:
a) an RNA oligonucleotide; or
b) a DNA oligonucleotide.

93.-102. (canceled)

103. The composition of claim 1, wherein the immunostimulatory oligonucleotide is SEQ ID NO: 2 or SEQ ID NO: 12.

104. The composition of claim 35, wherein the immunostimulatory RNA oligonucleotide is SEQ ID NO: 2 or SEQ ID NO: 12.

105. The method of claim Si, wherein the anti-viral agent is linked to the immunostimulatory oligonucleotide.

106. The method of claim 51, wherein the immunostimulatory oligonucleotide is:
a) an RNA oligonucleotide; or
b) a DNA oligonucleotide.

107. The method of claim 104 a), wherein the immunostimulatory RNA oligonucleotide is SEQ ID NO: 2 or SEQ ID NO: 12.

108. The method of claim 92 a), wherein the immunostimulatory RNA oligonucleotide is SEQ ID NO: 2 or SEQ ID NO: 12.
